(19) United States
(12) Patent Application Publication
(10) Pub. No.: US 2007/0163600 A1
(43) Pub. Date: Jul. 19, 2007

(54) User interface and head gear for a continuous positive airway pressure device

(76) Inventor: Leslie Hoffman, Tarzana, CA (US)

Correspondence Address: STETINA BRUNDA GARRED & BRUCKER, 75 ENTERPRISE, SUITE 250, ALISO VIEJO, CA, 92656, US

(21) Appl. No.: 11/649,674

(22) Filed: Jan. 4, 2007

Related U.S. Application Data
(60) Provisional application No. 60/758,151, filed on Jan. 11, 2006.
(60) Provisional application No. 60/793,704, filed on Apr. 20, 2006.

(51) Int. Cl. A62B 7/00; A61M 15/08

(52) U.S. Cl. 128/207.18; 128/200.24

(57) ABSTRACT

A user interface for a portable continuous positive airway pressure (CPAP) device comprises a gas delivery member releasably mountable to a manifold member. The CPAP device comprises a motor blower unit contained in a wearable vest which is connectable by a patient hose to the user interface. Alternative embodiments of the gas delivery member include a nasal mask or a pair of nasal prongs which are interchangeably mountable to the manifold member. Ball joints on opposing ends of the patient hose swivelably interconnect the patient hose to the user interface and to the motor blower unit. Cheek pads extend from opposing ends of the manifold member and are freely orientatable relative thereto. Six-way adjustable head gear stabilizes the user interface on the patient's face and comprises side straps and head straps which are pivotably joined to one another and which are adjustable lengthwise to fit a wide range of patient

### CROSS-REFERENCE TO RELATED APPLICATIONS

The present application claims priority to U.S. Provisional Application No. 60/758,151, filed on Jan. 11, 2006, and to U.S. Provisional Application No. 60/793,704, filed on Apr. 20, 2006, the entire contents of the each provisional application being expressly incorporated by reference herein. The present application is related to U.S. Utility Patent Application Ser. No. 11/128,552 entitled PORTABLE CONTINUOUS POSITIVE AIRWAY PRESSURE SYSTEM and filed on May 13, 2005, the entire contents of which is expressly incorporated by reference herein.

### STATEMENT RE: FEDERALLY SPONSORED RESEARCH/DEVELOPMENT

(Not Applicable)

### BACKGROUND

The present invention relates generally to patient ventilation systems and, more particularly, to a uniquely-configured user interface and fully-adjustable head gear as may be used with a portable continuous positive airway pressure (CPAP) device. The user interface and head gear are specifically adapted to provide enhanced patient mobility and comfort as a means to improve patient compliance with prescribed CPAP therapy.

Obstructive sleep apnea (OSA) is a serious health condition affecting as many as one in five adults. OSA is a breathing disorder characterized by a temporary collapse of the throat resulting in a pause in breathing during sleep. Each OSA episode can occur hundreds of times in a single night with each occurrence disrupting the patient's sleep or awakening the patient. Left untreated, OSA can lead to severe and even life-threatening consequences.

For example, the link between OSA and hypertension, stroke and heart failure is well-documented. Serious cases of OSA can result in sleep deprivation or insomnia which, over time, can result in moodiness, irritability, memory loss, poor judgment and an overall poor quality of life. Even further, patients suffering from OSA have a dramatically increased risk of traffic accidents and an increased mortality rate due to medical complications stemming from this disorder.

In severe cases of OSA, doctors commonly prescribe CPAP therapy wherein a constant flow of positively pressurized gas is supplied to the patient during sleep. The gas is typically pressurized to between 5 and 20 cm H₂O and is delivered to the patient's airway in order to hold the throat open and allow for uninterrupted breathing during sleep. Conventional CPAP devices typically include a blower unit connected by a patient hose to a mask. The mask acts as a nasal or oral interface and introduces pressurized gas into the patient's throat. The blower unit of conventional CPAP devices is typically powered by an electric motor which, due to noise, vibration and heat produced during its operation, must be mounted on a table or a stand located adjacent the patient's bed to avoid disrupting the patient's sleep.

Conventional CPAP patient hoses are flexible tubes typically provided in a standard six-foot length. The patient hose extends between the bedside blower unit and the mask which is mounted on the patient's head. Because of its long length and because the patient hose extends laterally or sideways from the patient to the blower unit, a sideways “torqueing” or pulling force is imposed by the patient hose on the mask. The torqueing or pulling by the patient hose results in poor sealing of the mask against the patient's face. In addition, the sideways pulling on the mask may also create pressure points against the patient's face and results in general patient discomfort.

For patients who use nasal prongs, the sideways tugging can cause irritation of the patient's nose due to the close-fitting engagement of the prongs with tender mucous tissue lining the patient's nostrils. For patients who use a nasal mask which seals around an exterior of the patient's nose, the tugging of the patient hose can prevent proper sealing of the mask and can also cause eye irritation as a result of pressurized gas leaking around the nose bridge of the mask and flowing into the patient eyes.

Another problem associated with the lengthy patient hose of conventional CPAP devices is the occurrence of condensation in the patient hose. Some conventional motor blower units operate at a relatively high temperature such that the pressurized gas produced thereby is typically heated. As the heated gas travels along the lengthy hose from the blower unit to the patient, the gas cools because the temperature of ambient air in the room is typically lower than the temperature of the pressurized gas. Moisture in the pressurized gas therefore condenses within the hose interior. During a period of use, this condensation can result in water buildup and the patient hose then becomes a breeding ground for bacteria resulting in colds and other health complications for the patient.

Closely related to the problem of tugging by the patient hose is a general lack of mobility associated with conventional CPAP devices. For patients who get up many times during the night, the patient hose acts as a restraint on movement as the patient is effectively tethered to the bedside blower unit. For those with active sleep patterns, the lengthy patient hose inhibits normal body shifting movements and turning from side-to-side to which the patient is accustomed such that the hose makes falling asleep difficult or prevents sleep altogether.

The above-mentioned problems associated with the patient hose are responsible in large measure for the generally low rate of compliance by patients who have started CPAP therapy. Other factors responsible for the low compliance rate include a general dislike of the medical-equipment appearance of a bedside CPAP device in a bedroom environment. Many patients simply have a general aversion to conventional CPAP devices.

As can be seen, there exists a need in the art for a user interface for ventilation systems such as CPAP devices wherein a variety of gas delivery members such as nasal prongs or a nasal mask may be interchangeably employed with the user interface to prevent irritation and other discomfort associated with continued use of a single type of gas delivery member. Furthermore, there exists a need in the art for a user interface allowing for greater freedom of movement of the patient and particularly at the patient's head during CPAP therapy but without the problem of the hose tugging on the mask as is commonly associated with conventional CPAP devices. In addition, there exists a need in the art for an improved head gear such as for use with a CPAP device and which has a wide range of adjustability to fit a wider range of patients with increased comfort to improve the compliance rate for CPAP therapy.

### BRIEF SUMMARY

The present invention specifically address and alleviates the above referenced deficiencies associated with conventional CPAP devices and other ventilation systems of the prior art. In one embodiment, a uniquely-configured user interface is specifically configured to allow the patient to select from nasal prongs or a nasal mask as the desired configuration for delivery of pressurized gas to the patient. Each of the nasal prongs and nasal mask includes a resilient, deformable flange extending about an inlet opening and which is configured to removably insertable into a corresponding pair of manifold outlet openings formed in a manifold member.

In its broadest sense, the user interface comprises the manifold member and the gas delivery member which is releasably coupleable to the manifold outlet openings. The pressurized gas flows from a motor blower unit of the CPAP device through a patient hose and into the manifold member. The pressurized gas then enters the manifold outlet openings and passes through the nasal prongs and/or nasal mask for delivery to the patient's airway. A pair of resilient flanges disposed on each of the nasal mask and nasal prongs allow for releasable attachment thereof to the manifold member for greater convenience in disassembly for cleaning and for substituting different types of gas delivery members at the patient's discretion.

The manifold member may be manufactured as a mating pair of symmetrically-formed shell portions which may be economically mass produced through a variety of suitable technologies such as by injection molding. The shell portions may be joined to one another along a longitudinal parting plane by any suitable means such as by using mechanical fasteners, adhesively bonding or sonically welding the shell portions together. The manifold inlet and outlet openings as well as exhaust ports are collectively defined by the mated shell portions. The exhaust ports are preferably aligned with the manifold outlet openings in order to direct the flow of exhaled CO₂ through the gas delivery member and out of the exhaust ports. The manifold member is also preferably configured with a small interior volume in order to minimize dead space and thereby improve the elimination of CO₂ into the atmosphere and to prevent rebreathing of the CO₂.

The user interface may be connected to the patient hose at the manifold member with at least one ball joint disposed on at least one of opposing hose ends of the patient hose. Advantageously, the ball joint is configured to swivelably interconnect the patient hose to the manifold member at the upper end and to the motor blower unit at the lower end. The CPAP device may be provided in a vest assembly arrangement similar to that described in greater detail in U.S. patent application Ser. No. 11/128,552 filed on May 13, 2005, or in the arrangement described in U.S. Provisional Application No. 60/793,589, filed on Apr. 4, 2006, the entire contents of both application being hereby incorporated by reference.

Each of the ball joints includes a ball member or hose fitting with at least one end thereof including a spherical overmold or a frusto-spherical ball portion for engaging a corresponding spherical undercut formed in a mating sleeve portion. The ball member may be provided in a single-ended version or in a double-ended version having opposing ball portions interconnected by a neck region. The ball portions may be mounted to conventional CPAP hose material which is typically flexible due to the inclusion of annular corrugations or bellows extending along the length of the patient hose.

When used with the vest-like CPAP device mentioned above, the patient hose extends generally vertically downwardly from the user interface and is generally centered along the patient's chest when the vest assembly of the CPAP device is worn over the patient's shoulders.

In this regard, the patient hose is a relatively short section and is therefore of relatively low mass in order to minimize gravitational and inertial forces acting upon the nasal mask or nasal prongs and to eliminate problems associated with lateral tugging or torqueing on the nasal mask or prongs as is common in conventional CPAP devices.

In an optional configuration, the patient hose may be comprised of a series of ball joints disposed end-to-end wherein each ball joint includes a sleeve member that is swivelably coupleable to an adjacent ball member. This arrangement allows for specific tailoring of the length of the patient hose as well as accommodating a wider range of motion of the patient's head without tugging by the patient hose on the mask. The patient hose can be provided in any desired length suitable for off-patient use of the CPAP device. For example, the CPAP device may be placed near the patient such as on a pillow in the patient's bed while the patient is sleeping. The flexibility provided by the ball joints and the low mass of the relatively short patient hose results in a high degree of movement for the patient without problems of air leakage around the mask or pressure points on the patient's face as are more commonly associated with conventional CPAP devices.

Resilient cheek pads may be included with the user interface and are preferably rotatably connectable thereto such as at opposing end portions of the manifold member. The cheek pads may be configured to anatomically conform to a variety of facial structures and assist in stabilizing the user interface in a comfortable position regardless of patient movement. The rotatable swivelable nature of the cheek pads is facilitated by mounting the cheek pads on a connector at each of opposing end portions of the manifold member. The connectors are releasably securable to the manifold member through a ball and socket joint wherein the socket is preferably sized to frictionally engage the ball such that the connector maintains any orientation set by the patient.

The cheek pads may be provided in a variety of sizes, shapes and configurations and may be fabricated using different materials. For example, the cheek pads may be formed of foam or deformable plastic to conform to the patient's face. Attachment and removal of the cheek pads from the connectors is facilitated through the use of mounting stems on the cheek pads which are adapted to engage corresponding apertures formed in each of the connectors. The cheek pads are sized in a thickness that provide a desired amount of offset spacing of side straps for head gear which supports the user interface on the patient's head. In addition, the check pads prevent the uncomfortable upward forcing of the nasal prongs into the patient's nostrils. The removable nature of the cheek pads also facilitates cleaning or replacement of worn cheek pads or interchangeability of cheek pads of one material (e.g., foam) with a cheek pad of a different material (e.g., plastic).

As was mentioned above, the head gear is adapted for supporting and stabilizing the user interface with improved comfort by providing six-way adjustability through the use of pivot joints and fastener material providing wide latitude in length adjustability of the straps that make up the head gear. In this regard, the head gear comprises left-side and right-side assemblies each of which includes a top head strap, a bottom head strap and a side strap pivotably connectable to one another at the pivot joint. Free ends of the top head straps are provided with complimentary hook and loop fastener material such that combined length of the top head straps is selectively adjustable to suit a particular patient. Likewise, free ends of the bottom head straps are adjustable through the use of hoop and loop fastener material in order to conform to the patient.

The side straps are connectable to respective sides of the manifold member and are each selectively adjustable in length using the hook and loop fastener material. Due to its wide range of adjustability both in pivoting and in strap length, the head gear provides comfortable support for the user interface regardless of movement by the patient. The head gear components are economically mass produceable of any suitable material including, for example, vinyl material which may be rapidly and efficiently produced in the head and side straps by die-cutting. In this regard, each of the side straps and head straps are configured with generally straight side sections in order to minimize material waste. Hook and loop material may be secured to the appropriate front or back sides of each of the side and head straps using any suitable mechanical attachment means (e.g., sewing) or by adhesively bonding, or any combination thereof.

### BRIEF DESCRIPTION OF THE DRAWINGS

These and other features of the present invention will become more apparent upon reference to the drawings wherein:

FIG. 1A is an elevational view of a patient wearing a portable CPAP device in a vest assembly arrangement and illustrating the interconnection of the vest assembly to a user interface with a patient hose;

FIG. 1B is an elevational view of the CPAP device in an alternative embodiment wherein a motor blower unit is contained within a tabletop housing and is connectable to the vest assembly by an extension hose;

FIG. 1C is a side view of the user interface mounted on a patient's head using head gear for supporting and stabilizing a nasal mask of the user interface;

FIG. 1D is an elevational view of the CPAP device wherein the vest assembly is shown supported on a pillow near the patient and wherein the vest assembly is interconnected to the user interface by the patient hose;

FIG. 2A is a side view of the user interface interconnected by the patient hose to a connector elbow and illustrating a pair of ball joints disposed at opposing hose ends of the patient hose;

FIG. 2B is an alternative embodiment of the patient hose illustrating an arrangement of a series of ball joints disposed end-to-end with each ball joint comprising a ball member rotatably and pivotally connected to an adjacent sleeve member;

FIG. 3 is a perspective view of a user interface comprising a manifold member having nasal prongs removably coupled thereto and further illustrating the range of motion of the ball joint;

FIG. 4 is a cross sectional view of the manifold member and ball joint as illustrated in FIG. 3;

FIG. 5 is a cross sectional view of the ball joint located at a lower end of the patient hose and illustrating the range of motion of the patient hose relative to the connector elbow;

FIG. 6 is a perspective view of the manifold member illustrating a pair of manifold outlet openings to which a gas delivery member (i.e., nasal mask or nasal prongs) may be releasably coupled;

FIG. 7 is an end view of the manifold member illustrating a plurality of exhaust ports formed therein;

FIG. 8A is an enlarged cross sectional view of the ball joint located at the lower end of the patient hose and illustrating the frusto-spherical ball portions located on opposing ends of the ball member and being interconnected by a neck portion;

FIGS. 8B-8C are cross sectional views of hose fittings provided with spherical overmolds and which are sized and configured to engage a spherical undercut formed in the sleeve portion of the connector elbow;

FIGS. 8D-8J illustrate the connector elbow having a bore formed laterally relative to a sleeve axis and which is adapted to receive a retainer pin for releasably connecting the connector elbow to a blower outlet fitting;

FIGS. 9A-9H illustrate the gas delivery member configured as the nasal prong and illustrating a deformable flange formed thereon for releasably coupling the nasal prong to a manifold outlet opening;

FIG. 10 is a perspective view of the user interface illustrating the gas delivery member in the nasal mask configuration;

FIG. 11 is a side view of the user interface having the nasal mask mounted to the manifold member with a pair of flanges;

FIG. 12 is a perspective view of the nasal mask illustrating a triangularly-shaped mask outlet opening designed to sealingly engage and to substantially envelope the patient's nose;

FIG. 13 is a perspective view of an outer portion of the pair of flanges for releasably coupling to the manifold outlet openings;

FIGS. 14A-14H are various view of the nasal mask illustrating a mask shoulder portion extending about a periphery of the mask outlet opening and including a notch for engaging the bridge of the patient's nose;

FIGS. 15A-15H show one of a pair of symmetrical upper and lower shell portions which, when secured together, collectively define the manifold member;

FIGS. 16A-16C show the swivelably rotatable connection of a pair of connectors to opposing end portions of the manifold member;

FIGS. 17A-17D illustrate a connector ball for engaging a socket formed in the manifold member and further illustrating a pair of apertures for releasably engaging a pair of cheek pads;

FIGS. 18A-18E show the cheek pad in one embodiment and illustrate a pair of pad mounting stems for releasably engaging the pair of aperture formed in the connector;

FIGS. 19A-19C show the cheek pad in an alternative embodiment including a plurality of through holes;

FIGS. 20A-20B illustrate head gear connected to the user interface and illustrating the gas delivery member of the user interface being configured as the nasal mask;

FIGS. 21A-21B illustrate the head gear attached to the user interface and illustrating the gas delivery member being configured as a pair of the nasal prongs;

FIG. 22 is a perspective view illustrating the head gear in its operational state and comprising the opposing side straps and top and bottom head straps for securing the user interface to the patient;

FIG. 23 is a plan view of either one of the left-side and right-side assemblies comprising a side strap, a top head strap and bottom head strap pivotally connected to one another;

FIG. 24A is a plan view of individual ones of the side strap and a head strap and illustrating pivot ends and free ends with hook and loop fastener material secured to the side and head straps;

FIG. 24B is a cross sectional view of a rivet as may be utilized in the pivot joint; and

FIG. 25 is a plan view of the head gear in an alternative arrangement comprising a pair of the side straps pivotally extending from each one of the left-side and right-side assemblies.

### DETAILED DESCRIPTION

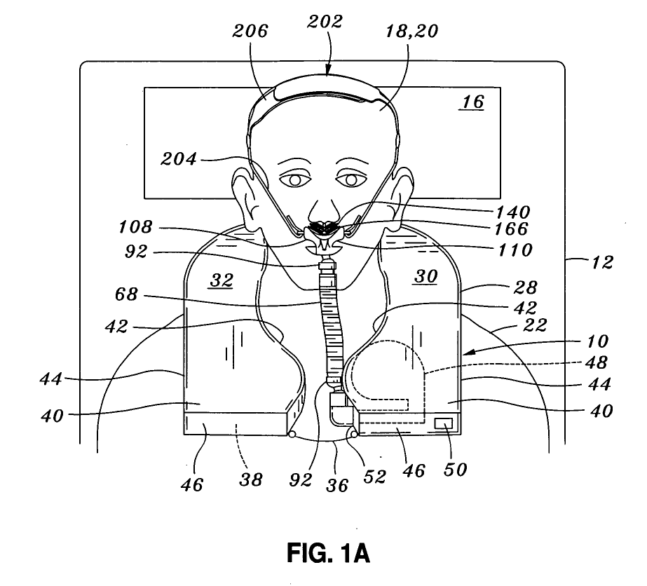

Referring now to the drawings wherein the showings are for purposes of illustrating preferred embodiments of the present invention and not for purposes of limiting the same, shown in FIGS. 1A, 1B and 1D is a continuous positive airway pressure (CPAP) device which is ergonomically-designed in a self-contained vest-like arrangement which may be worn (FIGS. 1A-B) or operated near a patient 18 (FIG. 1D). The figures further illustrate a user interface 108 as may be used with the portable CPAP device 10 or with alternative ventilation systems.

The user interface 108 may be provided in kit form and generally comprises a manifold member 110 and a gas delivery member 140. The gas delivery member 140 is providable in a variety of alternative configurations such as a nasal mask 142 or nasal prongs 166 and both are configured to be interchangeably mountable to the manifold member 110 at the patient's discretion. As can be seen in FIG. 1A, a ball joint 92 may be included between the user interface 108 and a patient hose 68. The patient hose 68 delivers pressurized gas from a motor blower unit 48 to the patient. The ball joint 92 provides increased flexibility in patient 18 movement as well as improving patient 18 comfort and the overall efficacy of CPAP therapy by reducing leakage at the nasal mask 142 or nasal prong 166.

The user interface 108 may further be provided with cheek pads 190 which are preferably freely orientatable to accommodate different facial structures. The cheek pads 190 maintain stability of the user interface 108 on the patient's 18 face. The figures further illustrate a uniquely-configured head gear 202 which provides six-way adjustability to fit a wide range of patients and which is selectively adjustable for comfort and is adaptable to alternative types of gas delivery member 140 (e.g., nasal mask 142 and nasal prongs 166).

As described in greater detail in U.S. patent application Ser. No. 11/128,552 entitled PORTABLE CONTINUOUS POSITIVE AIRWAY PRESSURE SYSTEM, the entire contents of which is incorporated by reference herein, the CPAP device 10 includes a vest assembly 28 comprising a first panel 30 and a second panel 32 interconnected by a collar portion 34. Importantly, the collar portion 34 has a reduced cross sectional area to better fit within the nape 26 of the patient's neck 24. The collar portion 34 interconnects the first and second panels 30, 32 which have a flared cross sectional shape. A motor blower unit 48 is housed in the first panel 30 and a control unit 60 is housed within the second panel 32. As disclosed in U.S. application Ser. No. 11/128,552, each of the first and second panels 30, 32 includes an inner side 38 which rests against the patient's chest and an exterior outer side 40. In the second panel 32, the control unit 60 contains control buttons 64 and a display 66 for regulating operation of the CPAP device 10. The control unit 60 is exposed to the outer side 40 of the second panel 32. A power source and/or battery pack 62 may also be contained within the second panel 32 for powering the CPAP device 10.

Referring still to FIG. 1A, a blower inlet 50 is preferably located adjacent the lower edge 46 of the first panel 30 near the motor blower unit 48 to serve as an air intake. A blower outlet 52 is likewise disposed adjacent the lower edge 46 but preferably opens toward an inner edge 42 of the first panel 30 to facilitate connection to the centrally-located patient hose 68 extending downwardly from the patient's head 20 when the CPAP device 10 is worn. The first and second panels 30, 32 may be removably connected to one another by means of a panel tie 36 or other suitable device for maintaining the orientation and location of the vest assembly 28 relative to the patient 18.

FIG. 1B illustrates an alternative embodiment of the CPAP device 10 wherein the motor blower unit 48 is housed in a table-top housing 12 as an alternative arrangement to containment of the motor blower unit 48 in the first panel 30 as shown in FIG. 1A. The CPAP device 10 shown in FIG. 1B further includes an extension hose 58 interconnecting the motor blower unit 48 to the first panel 30. The first panel 30 may contain a conduit 54 extending along a lower edge 46 thereof and having opposing conduit ends 56 for interconnection to the extension hose 58 and to the patient hose 68. The combination of the extension hose 58, the conduit 54 and the patient hose 68 provides fluid communication between the motor blower unit 48 and the user interface 108 for delivery of pressurized gas to the patient 18.

In the configuration shown in FIG. 1B, the vest assembly 28, when worn by the patient, decouples lateral forces otherwise imposed upon the gas delivery member 140 as a result of the mass of the extension hose 58 in its lateral or sideways orientation. In this manner, lateral tugging or torqueing forces imposed by the extension hose 58 are largely borne and dispersed by the vest assembly 28. The user interface 108 illustrated in FIGS. 1A and 1B improves patient 18 comfort as compared to traditional CPAP devices 10 in that the user interface 108 allows for the interchangeable mounting of a variety of configurations of the gas delivery member 140. For example, the gas delivery member 140 may be provided as the nasal mask 142 which envelopes and seals around the entire patient's nose. The gas delivery member 140 may also be provided as the pair of nasal prongs 166 which are insertable into and which seal inside the patient's nostrils.

The gas delivery member 140 may be configured in other configurations not shown including a full face mask which seals around the patient's nose and mouth, a nose cushion mask which seals over both of the patient's nostrils similar to the nasal mask, or an oral mask which fits into the patient's mouth. In this regard, the term “gas delivery member” is defined herein as any structure which terminates at the patient's nose and which is configured to deliver pressurized gas to the patient's airway. The interchangeability of these and other mask configurations is facilitated through the combination of manifold outlet openings 122 formed as a pair in the manifold member 110 and which are adapted to receive a corresponding pair of flanges 160 formed on each configuration of the gas delivery member 140.

FIG. 1A illustrates the user interface 108 having the nasal prongs 166 releasably secured to the manifold member 110 while FIG. 1B illustrates the gas delivery members 140 configured as the nasal mask 142 also releasably secured to the manifold member 110. A preferred embodiment of the user interface 108 is provided in kit form and includes both the nasal prongs 166 and a nasal mask 142 from which the user may select for use with the manifold member 110.

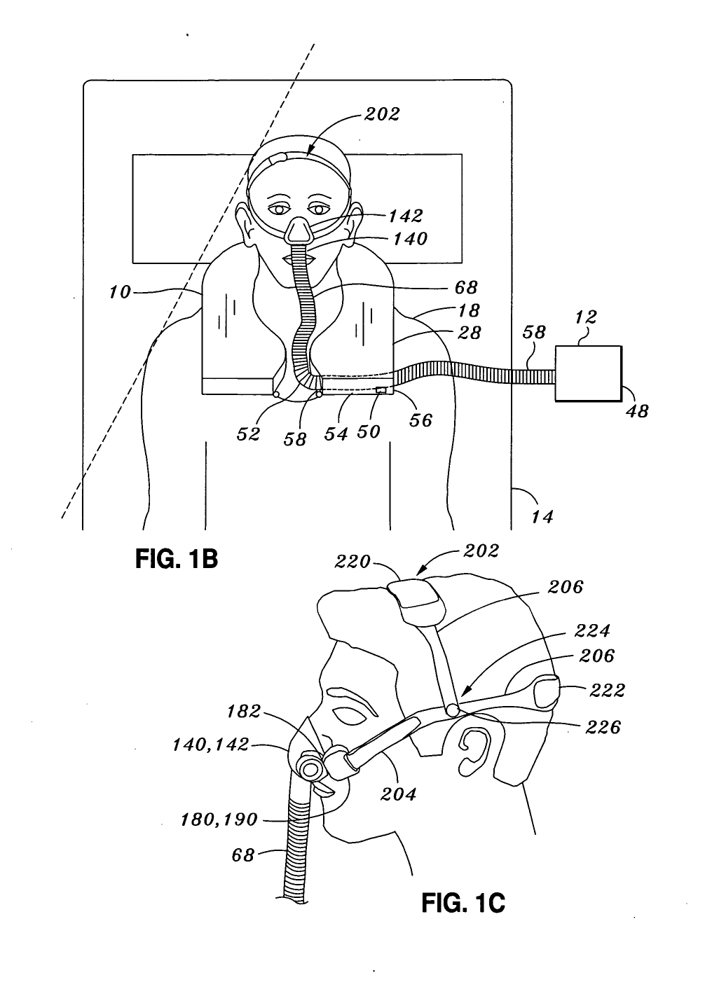

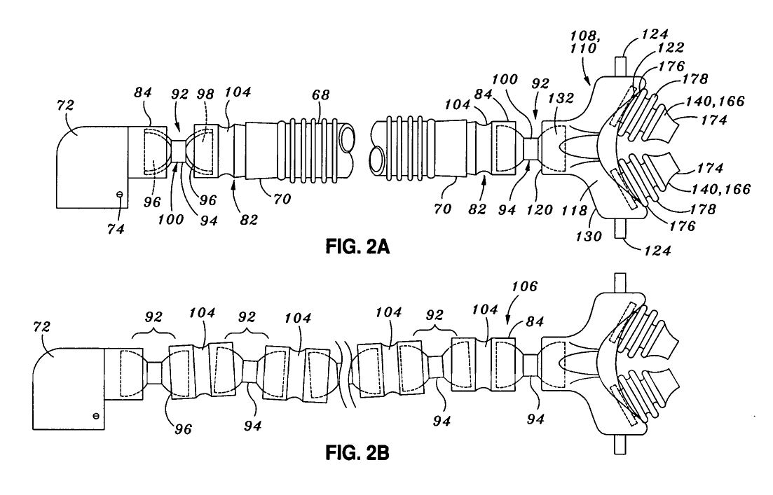

Referring now to FIGS. 2A through FIG. 7, the manifold member 110 can be seen as including a pair of manifold outlet openings 122 disposed in spaced relation to one another. The manifold outlet openings 122 are located on a side of the manifold member 110 opposite a manifold inlet opening 120 disposed in a center portion 132 of the manifold member 110 and which is connectable to the patient hose 68 such as via a ball joint 92 as will be described in greater detail below. The manifold member 110 is preferably hollow and defines an interior chamber 118 which acts as an air passageway 136 joining the manifold inlet opening 120 with the manifold outlet openings 122. In this regard, the manifold member 110 allows pressurized gas to flow through the manifold member 110 and into the patient 18 airway via the gas delivery member 140. As can be seen in FIG. 4, the pressurized gas moves along an inflow direction indicated by the arrow and exits a plurality of relatively small holes or exhaust ports 126 positioned opposite the manifold outlet openings 122.

During the exhalation phase of a breathing cycle, carbon dioxide (CO₂) is exhaled by the patient 18 and flows back through the manifold outlet openings 122 and exits through the exhaust ports 126. The relatively small volume occupied by the interior chamber 118 minimizes dead space within the manifold member 110 which improves CO₂ elimination through the exhaust ports 126 as the CO₂ exiting the patient's nostrils is able to flow directly out of the exhaust ports 126. Furthermore, the location of the exhaust ports 126 directly opposite the manifold outlet openings 122 and away from the patient 18 prevents rebreathing of the CO₂ and also directs noise produced by the exiting CO₂ away from the patient 18.

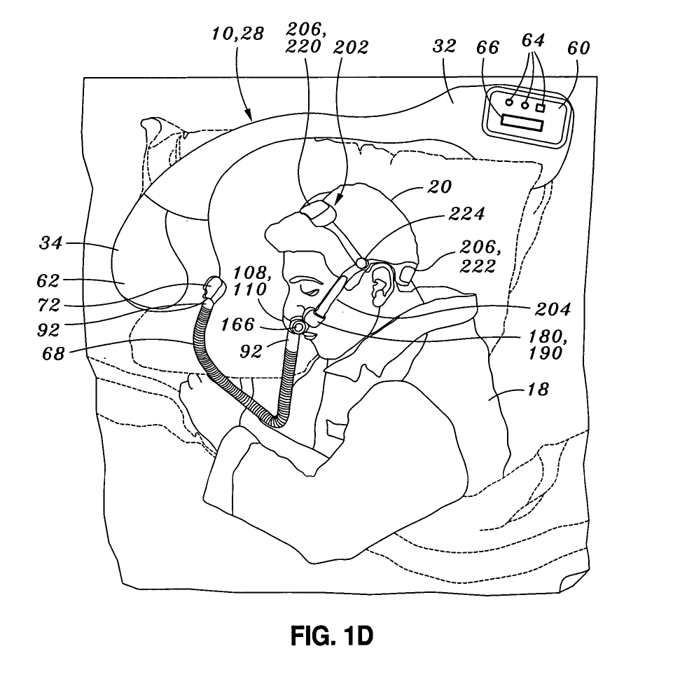

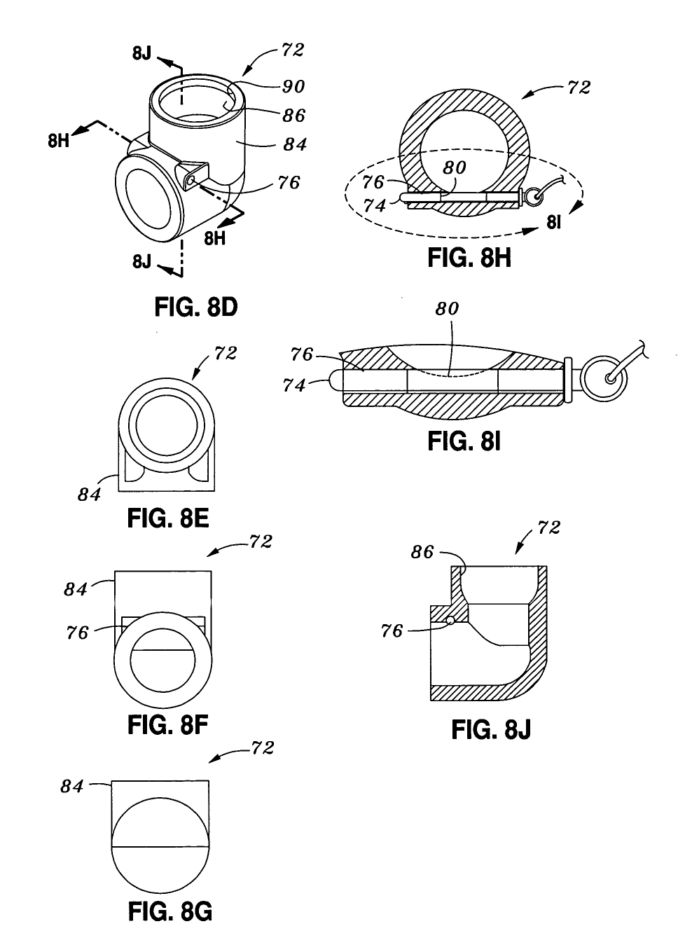

FIGS. 3, 4, 6 and 9A-9H illustrate removable connection of the nasal prongs 166 to the manifold member 110. Importantly, each of the nasal prongs 166 includes a flange 160 which is preferably resilient and therefore deformable in order to allow for removable insertion into one of the manifold outlet openings 122. The flange 160 and corresponding manifold outlet opening 122 are sized and configured such that the flange 160 is retained within the manifold outlet opening 122. As best seen in FIGS. 9A-9H, the nasal prong 166 includes a prong shoulder 176 disposed in spaced relation to the flange 160 to define a flange neck 162 which is also sized and configured complimentary to one of the manifold outlet openings 122. In this regard, the flange neck 162 is preferably of a smaller diameter than either the flange 160 or the prong shoulder 176 to facilitate engagement to the manifold outlet opening 122.

After squeezing the flange 160 to temporarily reduce its size to allow insertion into the manifold outlet opening, the flange 160 is released and returns to its full size such that the neck portion 100 is captured and is axially restrained by the flange 160 and the prong shoulder 176. The spacing between the prong shoulder 176 and the flange 160 is complimentary to the local thickness of the manifold member 110. Removal of the nasal prong 166 is effectuated by grasping an end section 174 while simultaneously squeezing and pulling the nasal prong 166 out of the manifold outlet opening 122.

In an exemplary embodiment shown in FIGS. 9A-9H, the nasal prong 166 includes a prong inlet opening 168 located adjacent the flange 160. The prong inlet opening 168 is in fluid communication with a prong outlet opening 170 by a prong air passage 172 extending therebetween. The end section 174 at the prong outlet opening 170 is shown in a tapered shape to anatomically conform and seal against the inner surface of the patient's nostrils. In this regard, the nasal prong 166 is also preferably provided with a slight oval configuration at the prong outlet opening 170 to better approximate the shape of a human nostril.

Importantly, each one of the nasal prongs 166 may include a bellows 178 between the prong shoulder 176 and the prong outlet opening 170. The bellows 178 can be defined as a localized radial expansion of the nasal prong 166 and which provides a means by which the prong outlet opening 170 may move off-axis relative to the axis of the prong inlet opening 168. The off-axis movement provided by the bellows 178 may facilitate differences in nostril spacing from one patient 18 to another and may further maintain the fit and seal of the nasal prong 166 in the patient's nostril during certain movements such as when the patient's head 20 rotates relative to the vest assembly 28. The bellows 178 facilitates axial movement of the prong outlet opening 170 by expanding and collapsing to provide some degree of flexibility in the length or distance between the prong inlet and outlet openings 168, 170 and thereby improve fitment of the nasal prongs 166 with patients having differing nostril lengths.

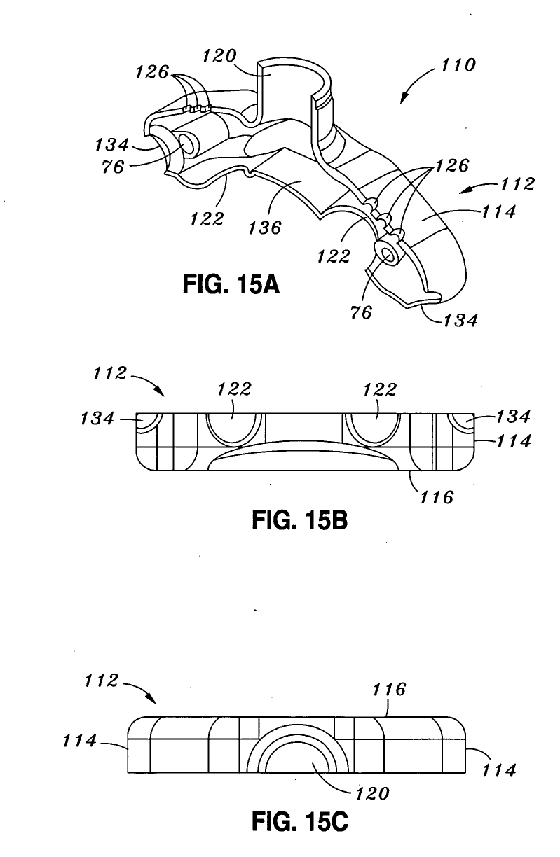

For the nasal mask 142 embodiment of the gas delivery member 140 shown in use on the patient 18 in FIGS. 1B and 1C and as shown coupled to the manifold member 110 in FIGS. 14A-14H, a pair of the flanges 160 are disposed on a mask outer portion 152 of the nasal mask 166. The general arrangement of the flange 160 upon the nasal mask 142 is similar to that which is described above for the nasal prong 166 illustrated in FIGS. 9A-9H wherein the nasal mask 142 includes a pair of neck portions 100 defined by the flange 160 and the mask outer portion 152. The neck portion 100 is preferably sized and configured complimentary to the manifold outlet openings 122.

The nasal mask 142 and each of the nasal prongs 166 is preferably formed of a resilient deformable material such as silicone to allow deformation of the flanges 160 for insertion into the manifold outlet openings 122 and to conform to the patient 18. The pair of flanges 160 formed on the nasal mask 142 define the mask inlet openings 144 which are in fluid communication with a triangularly-shaped mask outlet opening 146 best seen in FIG. 14A-14H. The mask outlet opening 146 is preferably sized and configured to substantially envelope and seal around the patient's nose.

Attachment and removal of the nasal mask 142 from the manifold member 110 is facilitated in a manner similar to that described above for attachment and removal of each of the nasal prongs 166 from the manifold member 110. Removal of the nasal mask 142 is facilitated by simply pull the nasal mask 142 in a direction away from the manifold member 110 until the resilient flanges 160 fold inwardly and slide through the manifold outlet openings 122. Retention of the nasal mask 142 within the manifold outlet openings 122 is by capture of the neck portion 100 within the manifold outlet openings 122.

As was earlier mentioned, the same attachment mechanism described above may be applied to gas delivery members 140 of any configuration and are not to be construed as being limited to the nasal prongs 166 and the nasal mask 142 illustrated in the figures. Each of the above-mentioned full face mask, nose cushion mask, and oral mask may be fitted with flanges 160 to facilitate releasable coupling to the manifold member 110. The user interface 108 therefore allows for selection of the mask best suited to the patient's particular physiology and medical condition. Due to their small size and low mass, nasal prongs 166 may be a most preferable configuration for the gas delivery member 140 but use of nasal prongs 166 is also dependent upon other factors such as whether the patient's mouth remains closed during CPAP therapy.

Advantageously, the ease of removing the gas delivery member 140 offers numerous other advantages including greater convenience in cleaning the gas delivery member 140 to prevent the growth of bacteria in the damp interior of the gas delivery members 140. Furthermore, the ability to interchange gas delivery members 140 may prevent the development of other health issues such as irritation, drying and cracking of the mucus membrane in the nostrils as a result of tight sealing of the nasal prongs 166. Temporarily stopping use of the nasal prongs 166 may allow such irritations to heal. In the meantime, a nasal mask 142 or other gas delivery member 140 may be used with the manifold member 110.

As indicated above, each of the nasal prongs 166 as well as the nasal mask 142 are preferably fabricated of a polymeric material such as silicone rubber which exhibits favorable characteristics including durability, resiliency, resistance to oxidation and the ability to conform to the patient's facial or nasal contours. However, any resilient material which is non-irritating and pliable to provide leak-free sealing may be utilized. Sealability to the patient 18 may be further enhanced by tailoring the local wall thickness 158 of the gas delivery member 140. For example, as shown in FIGS. 14c and 14h, the mask body is defined by the mask inner and outer portions 154, 152 which are interconnected by a side wall 114 extending around the mask body. The generally triangularly-shaped mask outlet opening 146 which envelopes the patient's nose is preferably provided with a rounded mask shoulder 154. The mask shoulder 154 curves slightly inwardly such that the edges of the mask shoulder 154 do not contact the patient's skin.

Conformal sealing of the nasal mask 142 is enhanced by tailoring the wall thickness 158 to increase flexibility in localized regions for better sealing and to improve comfort for the patient. FIGS. 14C and 14H of the nasal mask 142 illustrate an example of varying wall thicknesses 158. As can be seen, the nasal mask 142 has a reducing wall thickness 158 along a direction from the side walls 114 toward the mask shoulder 154 to facilitate sealing engagement to the patient's face. The reducing wall thickness 158 may be provided in a gradual taper or in step form as exemplified in FIG. 14C wherein the wall thickness 158 at the mask outer portion is approximately 0.100″ and reduces to a wall thickness 158 at the side wall 114 of 0.090″. Near the mask shoulder 154, the wall thickness 158 reduces further to approximately 0.050″ at the junction with the mask shoulder 154. The wall thickness 158 in the mask shoulder 154 itself has a final thickness of approximately 0.014″. As may be appreciated, the recited wall thicknesses are exemplary only and any range of thickness can be used.

Referring to FIGS. 14A-H, leak-free sealing of the nasal mask 142 against a variety of facial configurations may be still further enhanced by including a notch 148 in the mask outlet opening 146 as best seen in FIGS. 14A, 14E and 14F. The notch 148 is preferably configured to engage a ridge of the patient's nose. The geometry of the nasal mask 142 is preferably configured to minimize dead space which, as was previously mentioned, facilitates the removal of CO₂ during the patient's exhalation phase. The nasal mask 142 may be provided in a variety of sizes suitable to completely envelope the patient's nose with clearance between the tip of the nose to an inner surface of the mask outer portion 152.

The wall thickness 158 at the mask outer portion 152 adjacent the mask inlet openings 144 is preferably at its greatest in order to provide sufficient strength as may be required during removal and installation of the nasal mask 142. Likewise, the flanges 160 are preferably sized and configured to have a thickness that allows for insertion thereof into the manifold outlet openings 122 but are thick enough to prevent unintentional folding which may otherwise cause the flange 160 to disengage from the manifold outlet opening 122. The angular orientation of the flanges 160 relative to one another is preferably complimentary to that of the manifold outlet openings 122 on the manifold member 110. The spacing between the mask inlet openings 144 as defined by the flanges 160 is preferably complimentary to the spacing between the manifold outlet openings 122 in order to allow sealing engagement therebetween.

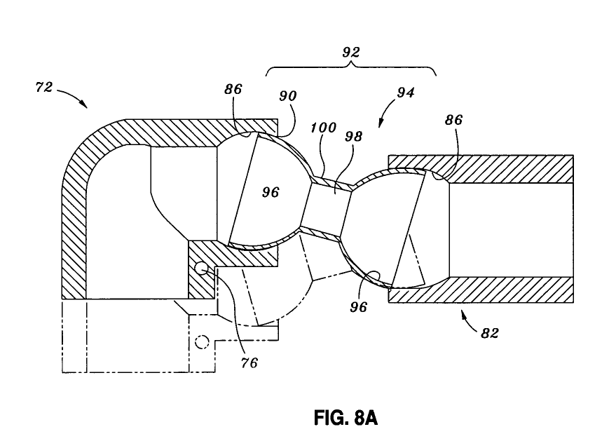

Referring to an embodiment illustrated in FIGS. 2-4, 6-7 and 11, the manifold member 110 can be seen as having a T-shaped configuration. As was earlier described, the manifold outlet openings 122 serve dual purposes of providing a directional path along which pressurized gas may flow from the manifold member 110 into the gas delivery member 140 (e.g., nasal mask 142 or nasal prong 166) as well as providing a means for releasably connecting the gas delivery members 140 to the manifold member 110 via the deformable flanges 160. The exhaust ports 126 are disposed directly opposite the respective ones of the manifold outlet openings 122 and provide a pathway for removal of CO₂. As best seen in FIGS. 4 and 7, the exhaust ports 126 are provided in equal number in each of the end portions 130 of the manifold member 110. Although three exhaust ports 126 are shown on both sides, any number may be provided.

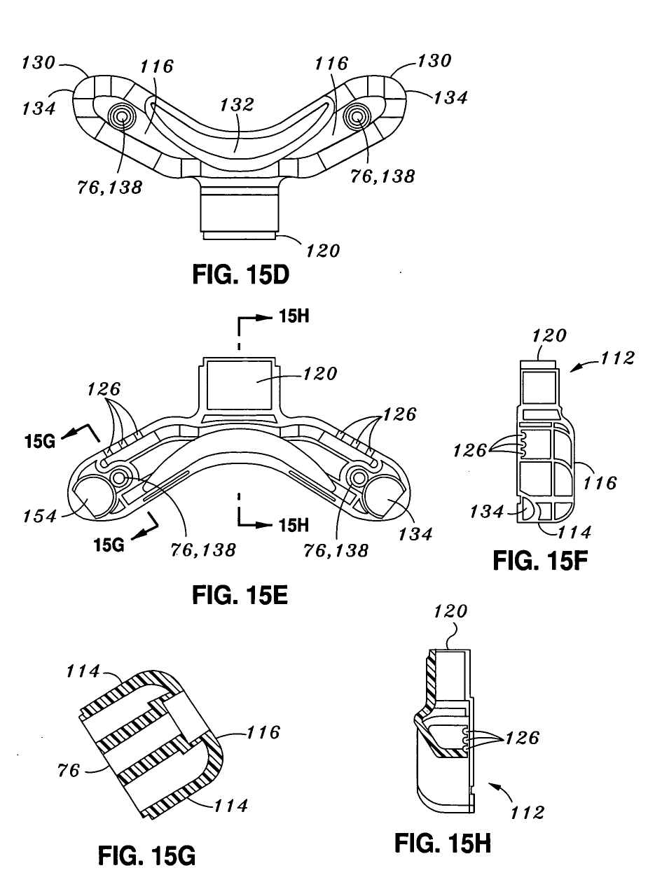

FIGS. 15A-H show the manifold member 110 in an alternative embodiment illustrating one of a pair of symmetrical upper and lower shell portions 112 which may be secured together in order to collectively define an entire manifold member 110. Each of the shell portions 112 can be molded of a suitable polymeric material such as polyethylene by injection molding or any other suitable molding process. The manifold member 110 preferably defines an interior chamber 118 occupying a minimal volume in order to eliminate dead space as was earlier mentioned

The embodiment shown in FIGS. 15A-H preferably includes an arch-shaped side wall 114 through which the manifold outlet openings 122 are formed in the end portions 130. As shown in FIG. 15D, a plurality of semi-circularly shaped exhaust ports 126 are formed in the side wall 114 along the parting plane and opposite the manifold outlet openings 122. Each of the end portions 130 is preferably angled away from the center portion 132 and may each be formed with a socket 134 to frictionally engage a connector 180 on each side of the manifold member 110. The connector 180 provides a means for attaching the head gear 202 to the user interface 108. As will be described in greater detail below, the socket 134 allows for swivelable and rotatable mounting of the connector 180.

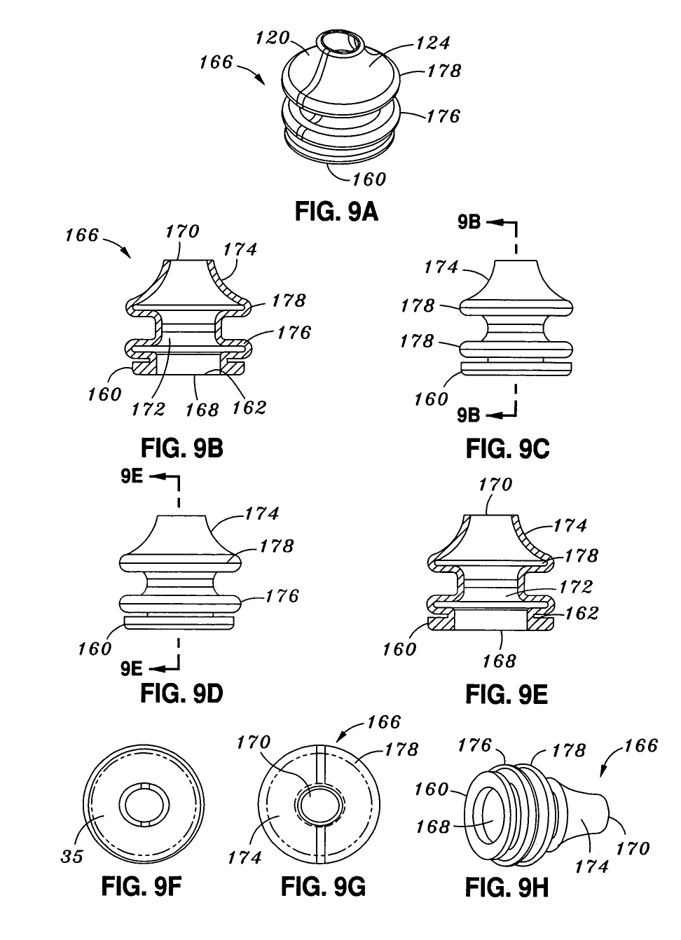

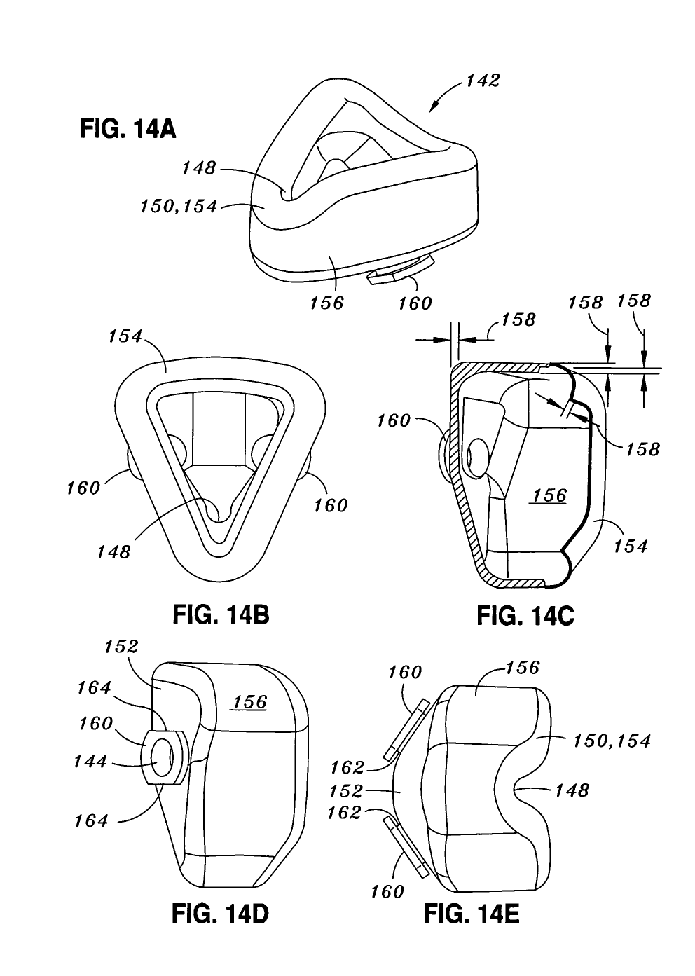

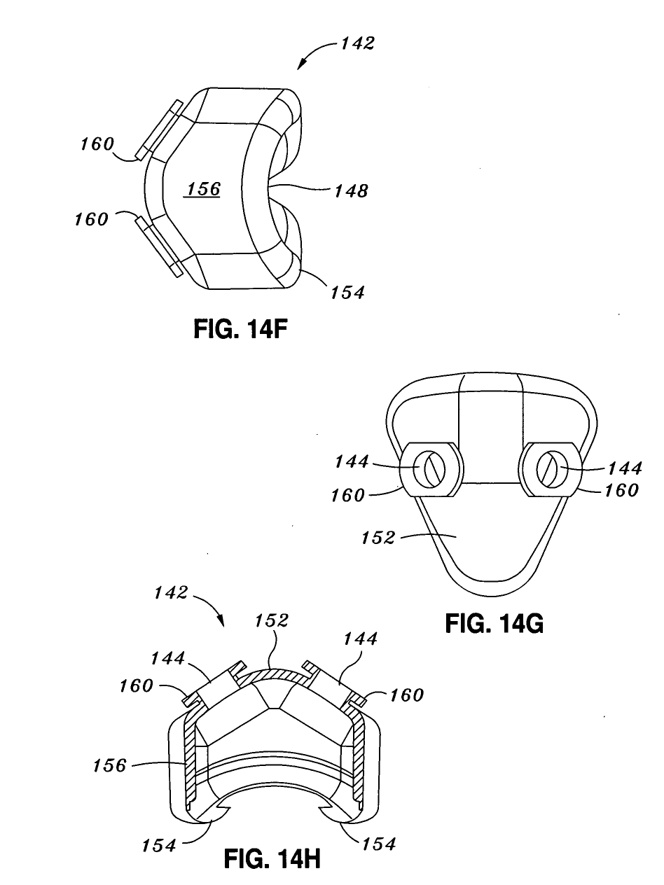

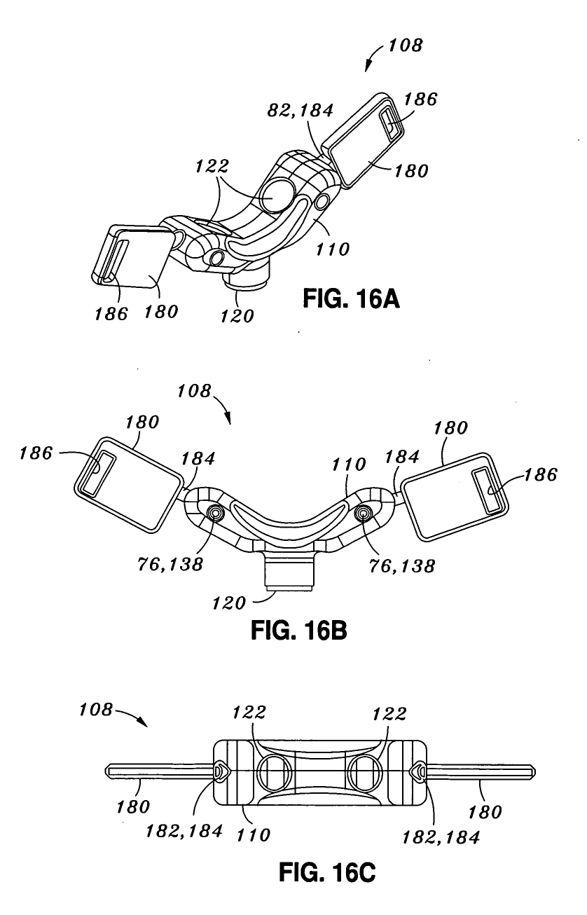

Alternatively, as shown in FIGS. 10-13, strap rings 124 may be swivelably connected to the end portions 130 of the manifold member 110 to provide a means for connecting to side straps 204 of the head gear 202. As can be seen in FIG. 10, the strap rings 124 are generally loop-shaped members extending laterally outwardly from each one of the end portions 130. The strap rings 124 are illustrated as being only swivelable connected to the manifold member 110 as compared to the arrangement shown in FIGS. 16A-C wherein the connectors are rotatable and pivotable to better accommodate movement of the patient's head 20.

Referring back to FIGS. 15A-H, each one of the symmetrical shell portions 112 is identically formed with a pair of sleeves or bores 76 for receiving mechanical fasteners. Counterbores 138 are preferably coaxially aligned with each of the bores 76 in order to allow for flush mounting of screws or rivets to provide a smooth surface along the outer wall 116 of the manifold member 110. In this regard, interconnection of the shell portions 112 to one another may be by other means such as by bonding along the outer edge of the side walls 114 by sonic welding, adhesive bonding or with interlocking features formed along the edges of the side wall 114 as illustrated in FIG. 15G. The manifold member 110 assembled from the shell portions 112 may define a general rectangular cross-sectional shape although any suitable shape may be used. Manufacturing of individual shell portions 112 provides advantages of economy and mass producibility as such parts can be quickly and easily manufactured at a low unit cost using a high-grade injection mold or other suitable manufacturing technology. However, those skilled in the art will recognize that the shell portions 112 can be fabricated in an asymmetrical configuration without departing from the spirit of the present invention as such configuration is expressly contemplated herein.

Referring to FIG. 2A, at least one ball joint 92 may be included at the user interface 108 and at the motor blower unit 48. As can be seen in FIG. 1A, the user interface 108 is fluidly interconnected to the motor blower unit 48 by means of the centrally-located patient hose 68 which extends downwardly away from the patient's face. The ball joint 92 may be removably connectable to opposing hose ends 70 of the patient hose 68 in order to allow greater patient 18 movement than would otherwise be achievable with a fixed connection between the patient hose 68 and user interface 108 or between the patient hose 68 and the motor blower unit 48. The ball joint 92 is disposable on at least one of the hose ends 70 of the patient hose 68 and provides improved freedom of movement of the user interface 108 relative to the motor blower unit 48.

In an embodiment best illustrated in FIGS. 3 and 4, the ball joint 92 may be configured as a generally hollow ball member 94 interconnected to at least one sleeve member 106 and/or the manifold member 110. The ball member 94 is comprised of opposing ball portions 96 which are interconnected by a neck portion 100 and having an air passage 98 extending therethrough to the allow the passage of pressurized gas from the patient hose 68 to the user interface 108. The central portion of the manifold member 110 is configured complimentary to the ball member 94 shown in FIG. 4. In this regard, the manifold inlet opening 120 preferably includes a spherical undercut 86 sized and configured complimentary to a frusto-spherical shape of the ball portion 96. Optionally or in addition to, an annular ridge 90 may be formed on an inner side 38 of the spherical undercut 86 in order to prevent axial pull-out of the ball member 94 from the manifold member 110. Even further, the spherical undercut 86 may be altogether eliminated with the retention of the ball member 94 into the manifold inlet opening 120 being provided solely by the annular ridge 90.

Interconnection of the ball member 94 to the patient hose 68 may likewise be facilitated by formation of a spherical undercut 86 in the patient hose 68 or, more preferably, by employing a hose fitting 82 as illustrated in FIG. 4. The hose fitting 82 is specifically configured to releasably engage the ball member 94 in a “snap-fit” manner to the spherical undercut 86 and/or annular ridge 90 formed in the manifold inlet opening 120. The hose fitting 82 includes a sleeve portion 84 which may include the optional spherical undercut 86 and/or annular ridge 90. On an exterior surface, the sleeve portions 84 may include an annular undercut 104 with the remainder of the hose fitting 82 having a tapering cross section to facilitate mating to the patient hose 68. The spherical undercut 86 is preferably sized to allow releasable engagement of the ball portions 96 into and out of the sleeve portions 84 for convenience in assembly and disassembly of the patient hose 68 from the user interface 108. FIG. 3 illustrates the extent to which the ball joint 92 facilitates relative movement between the patient hose 68 and the user interface 108.

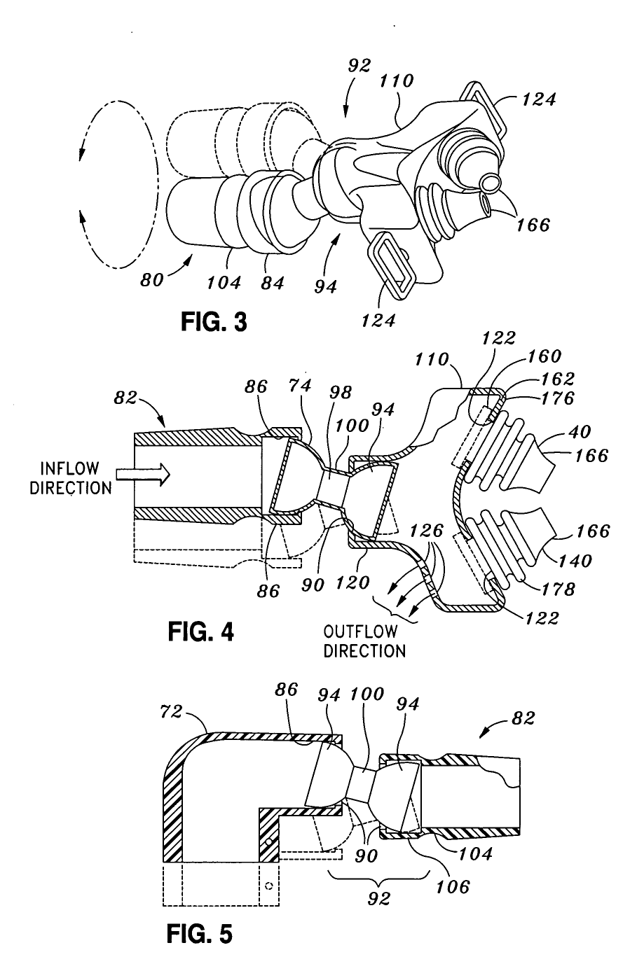

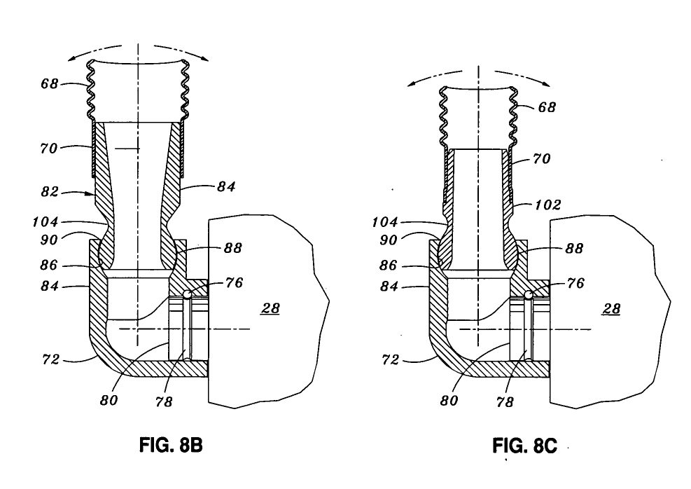

FIG. 5 illustrates the employment of a ball joint 92 at an opposite end of the patient hose 68 wherein the ball joint 92 interconnects the motor blower unit 48 to the patient hose 68. The arrangement and interconnectability of the motor blower unit 48 is similar to that which is described above with reference to the manifold member 110. In this regard, a hose fitting 82 may be employed in which the ball member 94 interconnects to the motor blower unit 48 at a connector elbow 72. As best seen in FIG. 8A, the spherical undercuts 86 formed in the hose fitting 82 and the connector elbow 72 provide a relatively wide range of motion between the patient hose 68 and the motor blower unit 48. However, it should be noted that ball joints 92 are not required at either end of the patient hose 68 and may be wholly omitted or a single ball joint 92 may be employed at only one of the hose ends 70.

As illustrated in FIGS. 8A-8J, the connector elbow 72 may provide a swivel capability to the patient hose 68. As best seen in FIGS. 8B and 8C, the connector elbow 72 forms a right-angle turn from the blower outlet 52 at the inner edge 42 of the first panel 30 of the vest assembly 28. A bore 76 is formed through the sleeve portion 84 of the connector elbow 72 as best seen in FIG. 81. The bore 76 extends into a portion of area occupied by a blower outlet fitting 80 to which the connector elbow 72 is secured. A complimentary annular groove 78 is formed in the blower outlet fitting 80 such that insertion of a retainer pin 74 as illustrated in FIG. 8I allows for removable coupling of the connector elbow 72 to the blower outlet fitting 80.

Referring to an additional embodiment best seen in FIGS. 8B and 8C, the hose fitting 82 may be generally configured as a single-sided ball member 94 wherein the sleeve portion 84 or the hose fitting 82 may be directly engaged to a hose end 70 of the patient hose 68. FIG. 8B illustrates a sleeve portion 84 having a relatively larger diameter than that which is shown in FIG. 8C such that patients hoses 68 of different diameters may be fitted to the CPAP device 10. Additionally, the hose fitting 82 illustrated in FIG. 8C is provided with a shoulder 102 against which the hose end 70 may be abutted. Each of the hose fittings 82 is preferably provided with an annular undercut 104 to facilitate a wider range of rotational movement of the hose fitting 82 relative to the connector elbow 72.

The hose fitting 82 shown in FIG. 8B may optionally be provided with a tapered inner surface in order to facilitate smooth flow of pressurized gas from the connector elbow 72 and into the patient hose 68. As can be seen in FIGS. 8B and 8C, the hose fittings 82 are provided with spherical overmold 88 portions which are functionally similar to the ball portions 96 formed on the ball members 94 as was earlier described. The spherical overmold 88 portions are configured to “snap-in” to the mating sleeve portions 84 and, specifically, to fit within the spherical undercut 86 formed in the sleeve portions 84 such as in the connector elbow 72.

The patient hose 68 may be provided in alternative embodiments. For example, the patient hose 68 may be provided as a short section of simple flexible hose as illustrated in FIG. 2A. Alternatively, the patient hose 68 may be formed as a series of ball joints 92 connected end-to-end as illustrated in FIG. 2B. The patient hose 68 illustrated in FIG. 2A is similar to conventional tubing used in ventilation therapy and may be comprised of conventional CPAP hose of standard size (i.e., diameter) and which is generally flexible due to the rings and/or bellows 178 formed into the patient hose 68. Preferably, the interior bore 76 of the patient hose 68 is smooth in order to eliminate restrictions on flow of the pressurized gas and to minimize noise produced thereby.

Such standard hoses are typically fabricated of polymeric material such as silicone rubber as described above. Advantageously, due to the detachable nature of the connector elbow 72 from the blower outlet fitting 80 upon removal of the retainer pin 74, the patient hose 68 may be easily disconnected from the vest assembly 28 to facilitate drying of the interior of the patient hose 68 as is recommended following each period of use. Disconnection of the patient hose 68 is further facilitated by the releasable nature of the ball joints 92 which are optionally disposed on opposing ends of the patient hose 68. Complete removal of the patient hose 68 facilitates drying thereof which may minimize the formation of mold or bacteria inside the patient hose 68. The detachable nature of the ball joints 92 and/or connector elbow 72 further facilitates separate washing of the patient hose 68 in soap and water in order to extend the life of the patient hose 68 and reduces the possibility of colds and other heath issues developing as a result of contaminants in the patient hose 68.

FIG. 2B illustrates an alternative embodiment of the patient hose 68 formed by the end-to-end connection of the ball joints 92. Each one of the ball joints 92 is comprised of a sleeve member 106 which is swivelably coupleable to a mating ball member 94. As was earlier mentioned, each of the ball members 94 is comprised of an opposing pair of frusto-spherical ball portions 96 separated by a neck portion 100. The entire ball member 94 is hollow to permit passage of pressurized gas. Each of the frusto-spherical ball portions 96 is receivable within a sleeve portion 84 of an adjacent sleeve member 106 as illustrated in Figure to be “snapped” into the spherical undercut 86. The end-to-end connection in series of the ball joints 92 permits tailoring of the overall length of the patient hose 68 and increasing the overall range of motion of the patient hose 68 as compared to that which is available with conventional CPAP hoses.

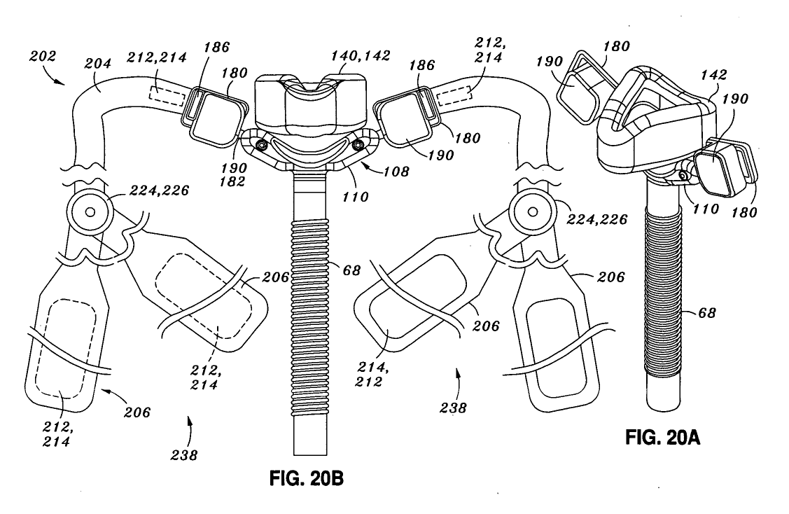

As can be seen in FIGS. 1C and 1D as well as in FIGS. 20-22, cheek pads 190 may be included with the user interface 108 and are preferably configured to be freely pivotable and rotatable within a wide range of motion. The cheek pads 190 are specifically adapted to engage the patient's cheeks in order to comfortably position the user interface 108 (e.g., nasal mask 142 or nasal prong 166) and stabilize the nasal mask 142 or nasal prongs 166. The cheek pads 190 may be provided in a predetermined thickness to maintain a spacing between the side straps 204 of the head gear 202 and the patient's face. The cheek pads 190 also prevent the nasal prongs 166 from being forced upwardly into the patient's nostrils.

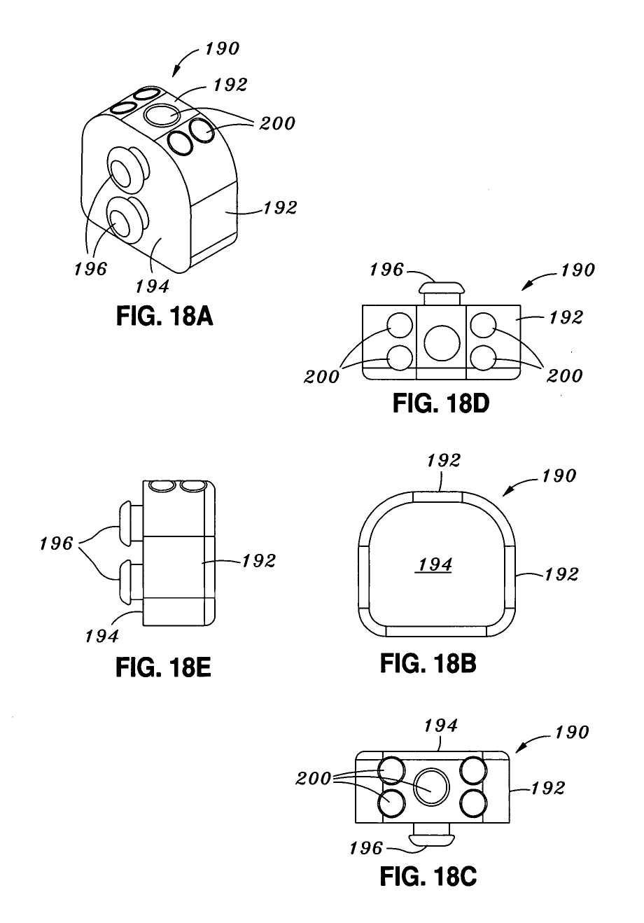

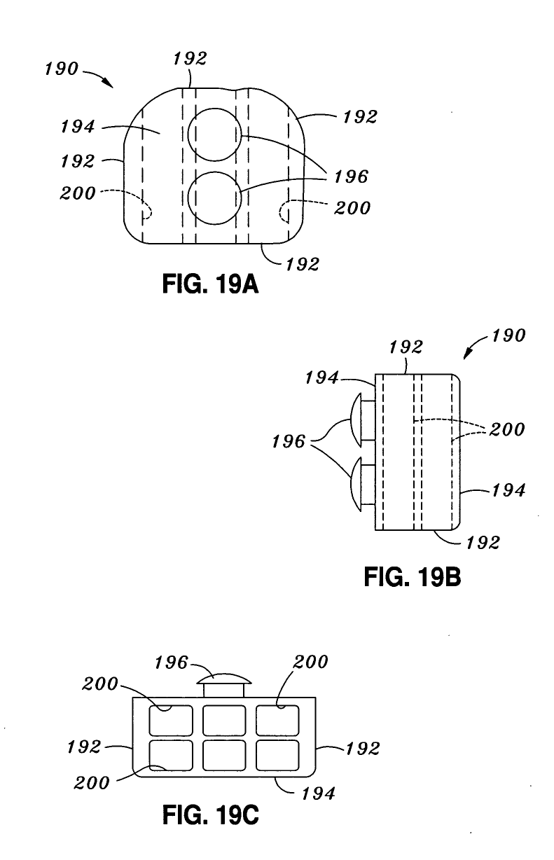

The cheek pads 190 may be provided in several alternative embodiments such as the embodiment formed of foam material illustrated in FIGS. 20A-B and 22 or the cheek pads 190 formed of plastic material as illustrated in FIGS. 18A-B, 19A-C and 21A-B. The foam embodiment of the cheek pad 190 may provide a relatively soft surface against the patient's face and therefore may be most suitable for pediatric patients or for those with sensitive skin. The plastic embodiment of the cheek pads 190 may optionally include through holes 200 to provide resiliency and therefore allow the cheek pad 190 to better conform to facial features. In addition, each of the cheek pad 190 embodiments, whether providable in foam or plastic, is configured to exhibit a desired stiffniess and geometry to allow comfortable resting against the patient's face.

The pad end wall 192 and side wall 194 as illustrated in FIGS. 18A-E and 19A-C are preferably radiused to maximize comfort. Material from which the cheek pads 190 are fabricated is also preferably non-reactive to the patient's skin or to natural oils, makeup, ointments or lotions. With regard to foam cheek pads 190, such material may better absorb perspiration as compared to cheek pads 190 fabricated of plastic. However, foam cheek pads 190 may also require more frequent replacement due to faster soiling from substances which could otherwise be cleaned from plastic cheek pads 190. Toward this end, each of the cheek pads 190 is preferably adapted to be removably connectable to the mask member due to the addition of mounting features such as pad mounting stems 196 illustrated in FIGS. 18a-e and 19A-C. Complimentary apertures 188 are formed in the connectors 180 which are swivelably and rotatably connectable to opposing end portions 130 of the manifold member 110.

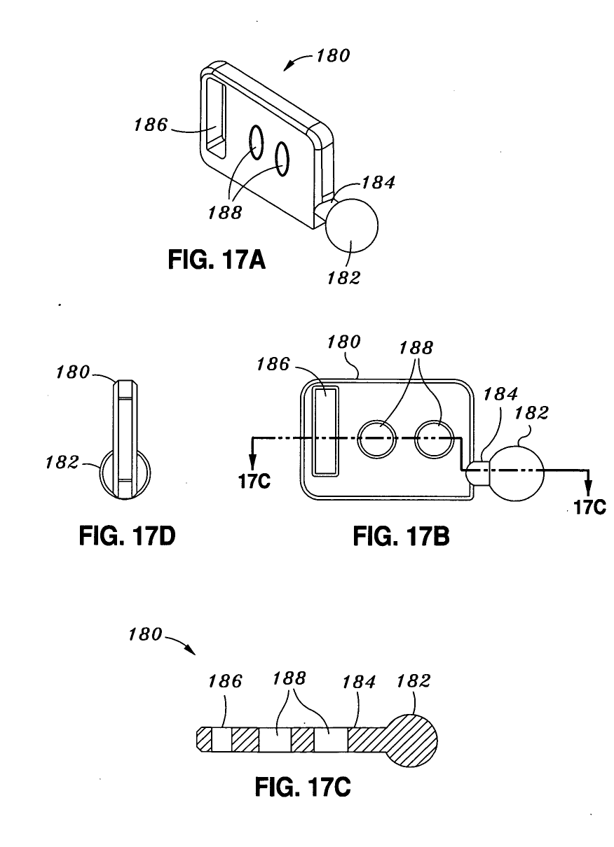

Referring to FIGS. 16A-16C and 17A-17D, the connector 180 can be seen as including the pair of apertures 188 which are sized and configured to receive the pad mounting stem 196. Each of the pad mounting stems 196 is comprised of a short shaft having an enlarged or flared portion on a free end of the shaft. The pad mount stems 184 are squeezed during insertion through the apertures 188 whereupon the flared portion expands to normal size, thereby capturing the shaft of the pad mounting stem 196 in the apertures 188. As can be seen in FIG. 16A-C, the connectors 180 extend laterally outwardly from each of the end portions 130. Each of the connectors 180 may include a connector ball 182 formed on an end thereof as can be seen in FIGS. 17A-D.

In a preferred embodiment, the connector ball 182 and socket 134 form a relatively tight frictional fit such that the connectors 180 maintain the orientation into which they are manually positioned. Advantageously, repositioning of the connectors 180 is therefore not required prior to each reinstallation of the user interface 108 on the patient's head 20. The connectors may be formed of any suitable polymeric material having sufficient stiffness for support the cheek pads 190 thereon. For example, a polyethylene material may be used for injection forming of the connectors 180. The connector ball 182 and socket 134 are preferably configured to be removable from one another by laterally pulling the connector 180 from the manifold member 110.

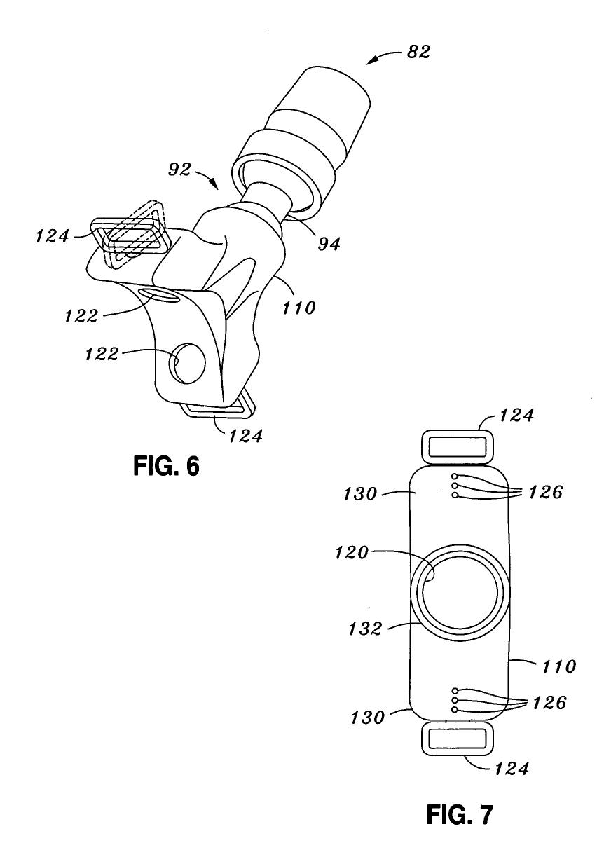

Connection of the head gear 202 to the manifold member 110 is facilitated by the slots 186 optionally formed in the connector 180 or by means of the strap ring 124 swivelably mounted to the manifold member 110 in an alternative embodiment as best seen in FIGS. 6 and 7. The head gear 202 itself provides a wide range of motion and is six-way adjustable by means of a set of top head straps 206, bottom head straps 206 and a pair of side straps 204 adjustably connectable to opposing sides of the user interface 108. As was mentioned above, the head gear 202 is adapted for mounting the user interface 108 to a patient's head 20 and may be with the CPAP device 10 described herein or with other ventilation systems. In this regard, the head gear 202 described herein can be used for securing a variety of user interface 108 configurations to a patient's head 20 due to its adjustability to fit a wide range of patients.

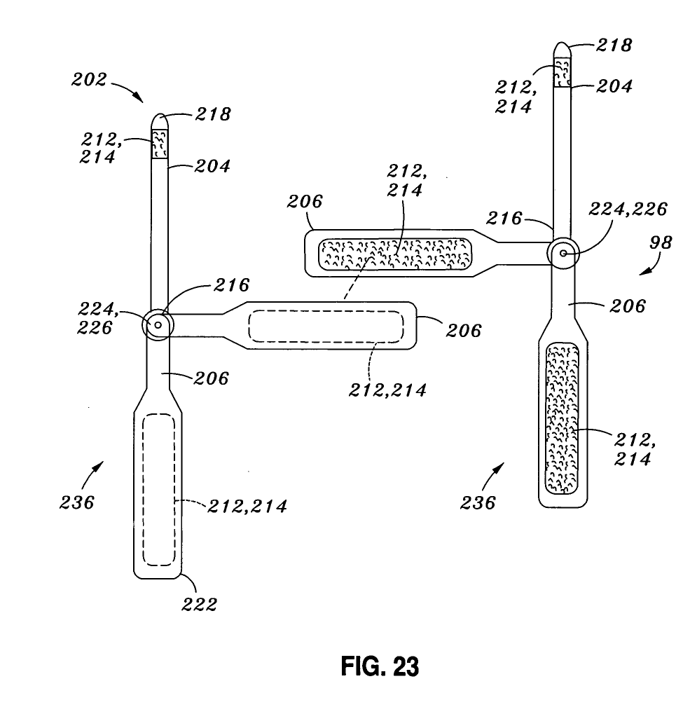

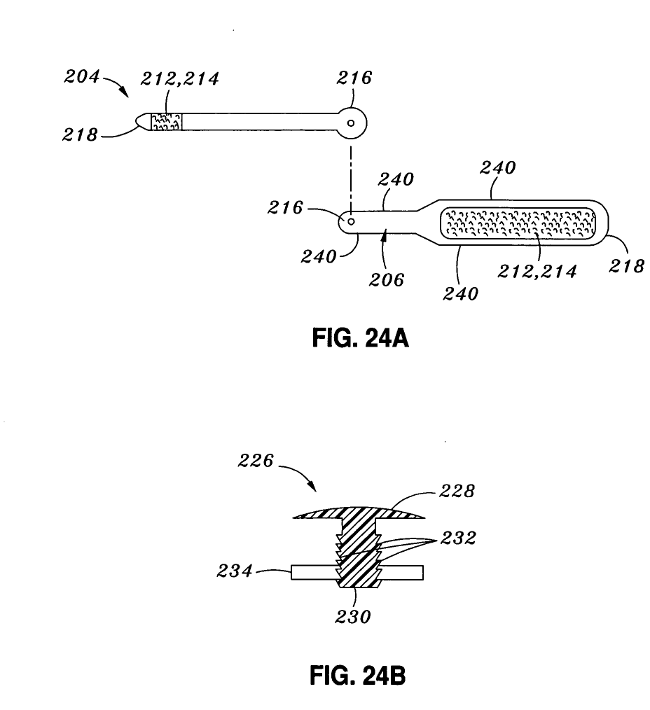

Referring to FIGS. 20B, 21B and 23, the head gear 202 is comprised of right-side assembly 236 including a top head 20 strap, a bottom head strap 206 and a side strap 204 pivotably connectable to one another at a pivot joint 224. Likewise, the head gear 202 also comprises a left-side assembly 238 including a top head 20 strap, a bottom head strap 206 and a side strap 204 also pivotably connectable to one another at a pivot joint 224. As best seen in FIG. 24C, each one of the head straps 206 and side straps 204 has a pivot end 216 and a free end 218. The free ends 218 of the side straps 204 are adjustably connectable to opposing sides of the user interface 108 such as via the connectors 180 illustrated in FIGS. 16A-C or the strap rings 124 illustrated in FIGS. 6 and 7. As best seen in FIG. 23, the free ends 218 of the top head straps 206 are adjustably connectable to one another such as by using complimentary mating fastener material 212 such as hook and loop material 214 (Velcro™). In this manner, the combined length of the top head straps 206 may be selectively adjustable relative to one another for extending over the top of the patient's head 20 and supporting the pivot joints 224 above the patient's ears.

Likewise, the free ends 218 of the bottom head straps 206 are adjustably connectable to one another in a manner similar to the top head straps 206 such as through the use of complimentary hook and loop material 212 formed on respective front and back sides 208, 210 of the mating bottom head straps 206. In this manner, the combined length of the bottom head straps 206 is selectively adjustable to. Preferably, the front side 208 of the side strap 204 includes both hook and loop material 212 such that the length of each one of the side straps 204 is selectively adjustable by adjusting the amount of overlap of the free end 218 of the side strap 204 with the remaining portion of the side strap 204. Alternative fastening means may be utilized for allowing selective adjustment of the top and bottom head straps 206 and the side straps 204. For example, mechanical fasteners including snaps, buckles (similar to that which is used in a belt for an article of clothing), or any other suitable adjustable fastening means may be used.

The pivoting relationship of the top and bottom head straps 206 relative to the side straps 204 provides a further measure of adjustability of the head gear 202. A hole is provided in the pivot ends 216 of each of the side straps 204 and head straps 206. A rivet 226 similar to that shown in FIG. 24D may be inserted through the three layers as illustrated in FIG. 24B such that the pivot ends 216 of the side and head straps 206 are captured between a head 228 of the rivet 226 and a washer 234 disposed on an opposite side of the layered straps. The washer 234 is axially engageable to on one of a plurality of radial ribs 232 formed on a shaft 230 of the rivet 226 such that a desired amount of compressive force may be applied to the straps to regulate resistance to pivoting.

In the interest of economy, it is contemplated that the side straps 204 are formed of any suitable flexible, planar material such as vinyl which may be preferably die-cut with straight sections 240 in order to minimize material waste and to facilitate mass production of the side straps 204 in an economical manner. The head straps 206 can be seen as having a narrow section and a wide section but with each section being formed as a straight section 210. Hook and loop material 214 may be applied to the front and/or back sides 208, 210 of the side straps 204 and head straps 206 such as by sewing, mechanical fastening, or by chemical means such as bonding with an adhesive.

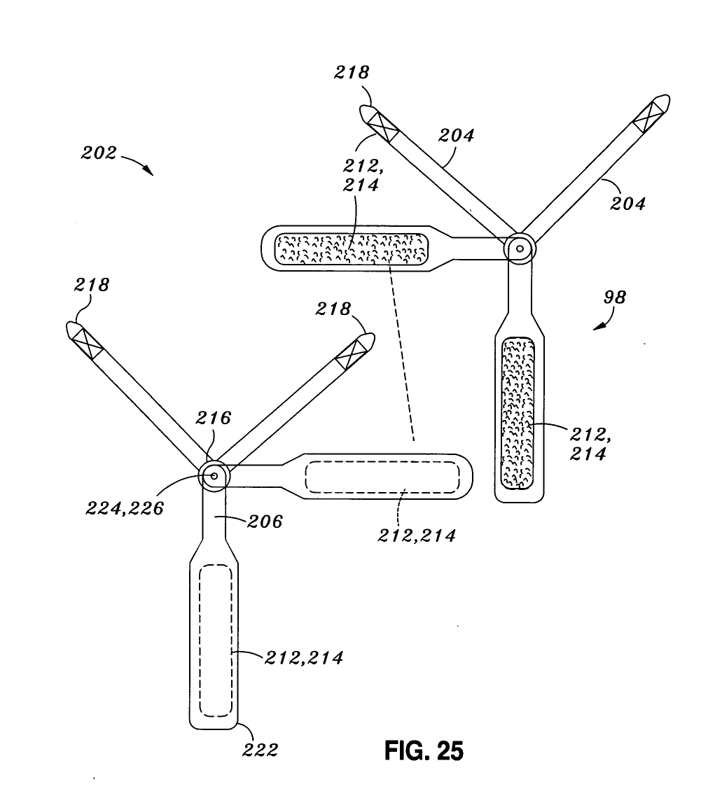

In an alternative embodiment of the head gear 202 illustrated in FIG. 25, each one of the right-side assemblies 236 and left-side assemblies 238 may include a pair of the side straps 204 as compared to the single side strap 204 included with each of the right-side and left-side assemblies 236, 238 illustrated in FIG. 23. The embodiment of the head gear 202 illustrated in FIG. 25 may be adaptable for use with relatively large mask configurations such as a full face mask described above. In such an embodiment, a lower one of the side straps 204 may be adjustably connectable to the manifold member 110 at opposing sides thereof while an upper one of the side straps 204 may be connected to a second set of connectors 180 or strap rings 124 mounted on an upper portion of the mask.

The operation of the user interface 108, patient hose 68 with optional ball joints 92, cheek pads 190 and head gear 202 in conjunction with the CPAP device 10 will now be described with reference to the drawings. The user interface 108 may be assembled by selecting from among several gas delivery member 140 configurations including the nasal mask 142 and the nasal prongs 166 described above. For releasable coupling of the nasal prongs 166 to the manifold member 110, the manifold outlet openings 122 are first located as can be seen in reference FIG. 6. Flanges 160 on each of the nasal prongs 166 are then slightly deformed in order to allow passage of the flange 160 through the manifold outlet opening 122. Upon release, the flange 160 expands to its original size such that the neck portion 100 of the nasal prong 166 occupies the manifold outlet opening 122.

The nasal prong 166 is captured by the flange 160 on one side of the manifold outlet opening 122 and the prong shoulder 176 on an opposing side of the manifold outlet opening 122. The opposite nasal prong 166 is installed into the remaining manifold outlet opening 122 in the same manner. Installation of the nasal mask 142 is accomplished in a similar fashion wherein the resilient material of the mask is compressible such that the flanges 160 may be squeezed for passage through the manifold outlet openings 122. Alternative mask configurations other than the nasal prongs 166 and nasal mask 142 may be coupled and decoupled to the manifold member 110 using flanges 160 in the same manner as described above.

The patient hose 68 connecting the user interface 108 to the motor blower unit 48 may be assembled by “snap-fitting” a ball joint 92 on at least one of opposing hose ends 70 as can be seen in FIG. 2A. The frusto-spherical ball portions 96 located on opposing ends of each of the ball members 94 are slidably inserted into the adjacent sleeve portion 84 of the hose fitting 82 and/or the manifold inlet opening 120 as best seen in FIG. 4. A ball joint 92 may likewise be connected to the hose end 70 on the opposite end of the patient hose 68 by snap-fitting the respective ball portions 96 into the hose fitting 82 and/or the connector elbow 72 as best seen in FIG. 5.

An alternative arrangement for the ball joint 92 at the hose end 70 adjacent the motor blower unit 48 can be seen in FIGS. 8B and 8C wherein the hose fitting 82 includes a ball portion 96 having a spherical overmold 88 on one end and a sleeve portion 84 on an opposing end. This single-sided version of ball member 94 is limited to pivotable motion at the interface between the connector elbow 72 and the hose fitting 82.

The patient hose 68 itself may be fabricated as a short section of standard tubing as used in conventional CPAP devices 10 and which is typically provided with a series of annular corrugations or bellows to provide some degree of flexibility to the patient hose 68. As best seen in FIG. 2B, the patient hose 68 may be constructed as a series of end-to-end ball joints 92 comprised of a plurality of the ball members 94 joined to adjacent sleeve members 106 to provide a greater degree of flexibility and the ability to tailor the overall length of the patient hose 68. Assembly of the patient hose 68 illustrated in FIG. 2B is similar to that which is described above with reference to interconnection of the ball joint 92 between the patient hose 68 and the manifold member 110 as illustrated in FIG. 2A.

The user interface 108 may be provided in kit form including a nasal mask 142 and a pair of nasal prongs 166 allowing the patient 18 to interchange the gas delivery members 140 at the patient's discretion. Advantageously, the ability to interchange the nasal prongs 166 with the nasal mask 142 may prevent the development of health issues normally associated with repeated use of a single type of gas delivery member 140. Additionally, the ease with which the nasal prongs 166 may be interchangeable substituted with the nasal mask 142 increases the convenience in cleaning and replacing components of the user interface 108 as they wear out.

The user interface 108 kit may further include a variety of patient hoses 68 provided in varying diameters and lengths. For example, in wearing of the vest assembly 28 as illustrated in FIGS. 1A-B, a short section of the patient hose 68 is required. If the vest assembly 28 is not worn by the patient 18 but is instead placed near the patient's head 20 such as on a pillow 16 while the patient 18 is sleeping as illustrated in FIG. 1D, a longer section of patient hose 68 may be more suitable. The latter scenario may be facilitated by using a patient hose 68 having a length of approximately 21 inches while the patient hose 68 in the worn configuration may be provided in a length of approximately 8 inches although the patient hose 68 may be provided in any length.

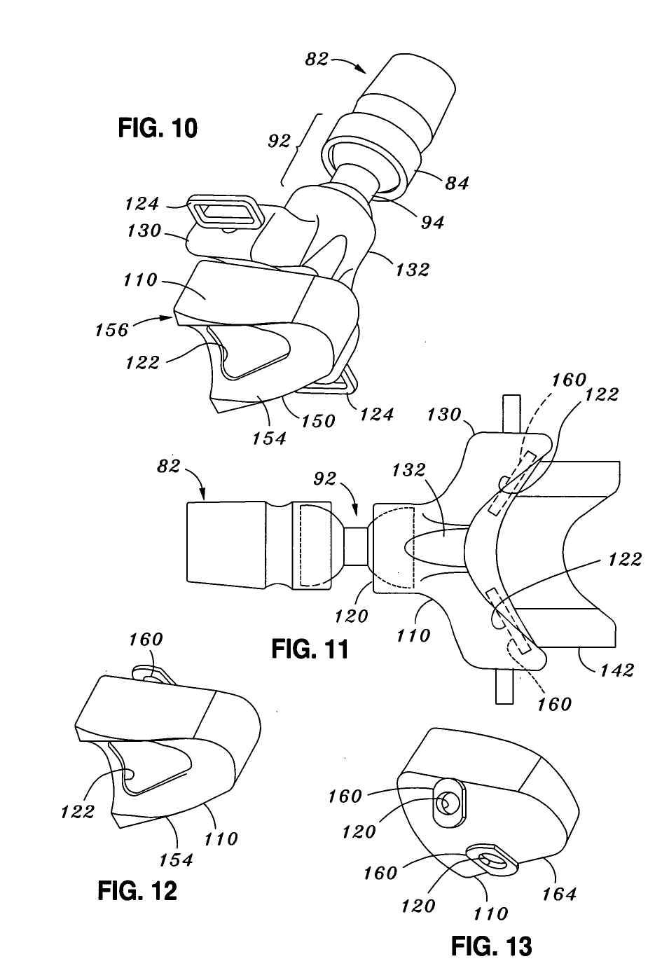

As illustrated in FIGS. 16A-C, if connectors 180 are included with the user interface 108, their attachment to the manifold member 110 is facilitated by insertion of the connector ball 182 into the corresponding socket 134 in the end portions 130 of the manifold member 110. However, the connectors 180 may be altogether eliminated with head gear 202 being simply secured to the strap ring 124 as illustrated in FIGS. 10 and 11. The ball joint provides additional flexibility in adjusting the connector 180 and, hence, the cheek pad 190, into any desired orientation.

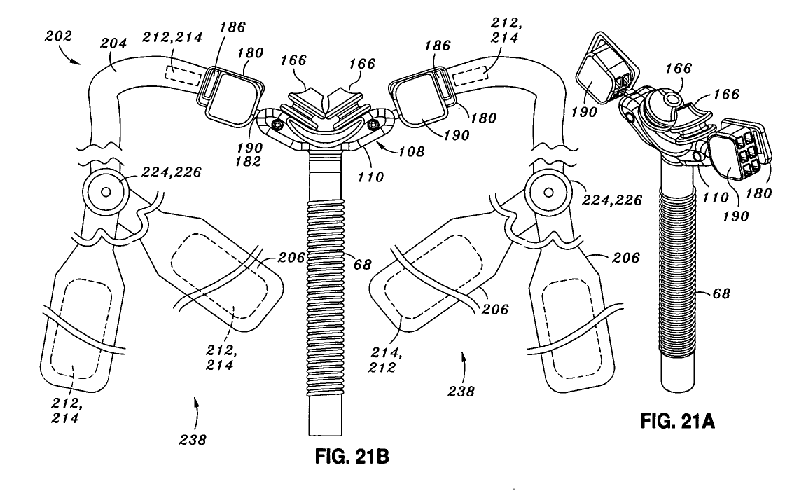

Cheek pads 190 material and configuration may be selected depending on whether the patient 18 desires a relatively soft foam material or a relatively harder but still deformable cheek pad 190 fabricated from plastic. As illustrated in FIGS. 20A-B, the cheek pads 190 of foam construction may provide a softer interface to the patient's skin. On the other hand, FIGS. 21A-B illustrate the installation of cheek pads 190 fabricated of plastic material and including through holes 200 to increase conformability.

Regardless of the particular configuration or material of the cheek pads 190, interconnection to the connectors 180 is facilitated by insertion of the pair of pad mounting stems 196 into the pair of corresponding apertures 188 formed in the connectors 120. Removal of the cheek pads 190 for replacement and/or interchangeability with a different configuration is easily accomplished by disengaging the pad mounting stem 196 from the apertures 188 and separating the cheek pads 190 from the connectors 180.

The head gear 202 may be connected to the user interface 108 in a manner similar to that illustrated in FIGS. 20a-b, 21A-B and 22. More specifically, the side straps 204 on each of the left-side and right-side assemblies 238, 236 may be inserted at their free ends 218 through corresponding slots 186 formed in each of the connectors 180. Hook and loop material 212 located on at least one of front and back sides 208, 210 of the side straps 204 allows adjustment in length to suit the patient 18.

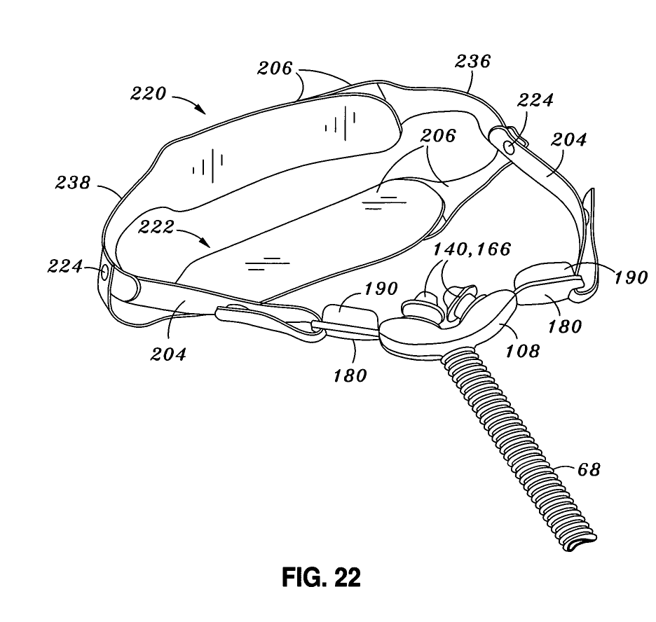

Referring now to FIGS. 22, 23, 24A and 24B, the top head strap assembly 220 is comprised of the top head straps 206 from the left-side assembly 238 and right-side assembly 236 are connected together in a similar fashion as is the bottom head strap assembly 222. FIG. 22 illustrates each of the top head strap 206 and bottom head strap 206 assemblies in the connected arrangement. The head gear 202 may then be fitted to the patient's head 20 on a trial-and-error basis by first locating the top head strap assembly 220 so that it extends over the top of the patient's head 20 with the bottom head strap assembly 222 wrapping around the lower portion of the patient's head 20 near the nape 26 of the neck 24.

Referring to FIG. 1C, each of the left-side and right-side assemblies 236, 238 is adjusted such that the pivot joints 224 are located approximately near the patient's ear although other locations are contemplated. Variations in the locations of the top head strap assembly 220 can be facilitated by pivoting the top and bottom head straps 206 relative to the side strap 204 so that the head gear 202 fits comfortably over the patient's head 20 with no pressure points. The head gear 202 is also preferably adjusted to provide sealing fitment of the selected nasal prongs 166 and/or nasal mask 142 without undue pressure against the patient 18. Simultaneous with the above-described adjustment of the side straps 204 and head 20 strap, the connectors 180 are pivotally and rotatably orientatable to position the side strap 204 into a comfortable location with the cheek pads 190 resting on each side of the patient's face. Optionally, where the gas delivery member 140 is configured as a full face mask requiring support at the upper portions, head gear 202 similar to that illustrated in FIG. 25 may be used and adjusted in a similar manner.

Connection of the patient hose 68 to the vest assembly 28 may be facilitated by first inserting the blower outlet fitting 80 into the connector elbow 72 in a manner similar to that shown in FIGS. 8B-C. The retainer pin 74 may be inserted into the bore 76 of the connector elbow 72 such that the retainer pin 74 engages a portion of the annular groove 78 in the blower outlet fitting 80. This arrangement allows for rotation of the connector elbow 72. Disassembly of the patient hose 68 from the vest assembly 28 is facilitated by removal of the retainer pin 74 from the bore 76 and sliding the connector elbow 72 off the blower outlet fitting 80. The patient hose 68 and user interface 108 may washed and hung to air dry after each use.

The vest assembly 28 may be either worn over the patient's shoulders 22 as shown in FIGS. 1A-B or the vest assembly 28 may be placed near the patient 18 in a manner illustrated in FIG. 1D. The high degree of flexibility provided by the ball joints 92 and/or patient hose 68 results in a greater freedom of movement for the patient 18 during sleep which prevents entanglement of the hose and therefore reduces the chances of sleep arousal. The added flexibility provide by the ball joints 92 and/or patient hose 68 also prevents air leakage at the gas delivery member 140 such that the efficacy of CPAP therapy provided by the vest assembly 28 is greatly enhanced relative to conventional bedside-mounted CPAP devices 10 having long, heavy hose which tug on the user interface 108.

Additional modifications and improvements of the present invention may also be apparent to those of ordinary skill in the art. Thus, the particular combination of parts described and illustrated herein is intended to represent only certain embodiments of the present invention and is not intended to serve as limitations of alternative devices within the spirit and scope of the invention.

What is claimed is:

1. A user interface adapted for delivering gas under pressure from a motor blower unit to a patient, the user interface comprising:
a manifold member having a pair of manifold outlet openings; and
a gas delivery member having a pair of inlet openings configured to be releasably coupleable to the pair of manifold outlet openings;
wherein:
the gas delivery member is configured as one of a nasal mask and a pair of nasal prongs, the nasal mask and nasal prongs being interchangeably connectable to the manifold member.

2. The user interface of claim 1 wherein:
the nasal mask and the pair of nasal prongs include a pair of flanges extending about a periphery of respective ones of the inlet openings;
the flanges being configured to be removably insertable into the manifold outlet openings.

3. The user interface of claim 1 wherein:
the nasal mask includes a mask shoulder extending along a periphery of the mask outlet opening;
the mask shoulder being configured to substantially sealingly engage a patient's face.

4. The user interface of claim 3 wherein:
the nasal mask includes side walls extending outwardly from the mask shoulder;
the side walls having a reducing wall thickness along a direction from the side walls to the mask shoulder such that the mask shoulder is substantially conformable to the patient's face.

5. The user interface of claim 1 further including:
a patient hose having opposing hose ends and being extendable between the manifold member and the motor blower unit; and
a ball joint being disposable on at least one of the hose ends and being configured to swivelably interconnect the patient hose to at least one of the manifold member and the motor blower unit.

6. The user interface of claim 1 further including at least one cheek pad removably coupleable to and extendable laterally outwardly from the manifold member and being selectively orientatable relative thereto.

7. A portable continuous positive airway pressure (CPAP) device, the CPAP device comprising:
a motor blower unit disposable within a wearable vest assembly, the motor blower unit being configured to produce pressurized gas;
a user interface configured to be coupleable to a patient for delivery of the pressurized gas to the patient's airway;
a patient hose having opposing hose ends and extending between the user interface and the motor blower unit; and
a ball joint disposable on at least one of the hose ends and being configured to swivelably interconnect the patient hose to at least one of the user interface and the motor blower unit.

8. The CPAP device of claim 7 wherein the patient hose is comprised of a series of the ball joints disposed in end-to-end arrangement.

9. The CPAP device of claim 8 wherein each one of the ball joints comprises a sleeve member swivelably connectable to a ball member.

10. The CPAP device of claim 7 wherein each one of the ball members includes an opposing pair of ball portions interconnected by a neck portion.

11. The CPAP device of claim 7 further including:
a connector elbow configured to be swivelably connectable to the ball joint at the hose end adjacent to the motor blower unit;
wherein:
the motor blower unit includes a blower outlet fitting configured to be removably engageable to the connector elbow and being rotatable relative thereto.

12. A user interface adapted for delivering gas under pressure from a motor blower unit to a patient, the user interface comprising:
a hollow manifold member formed of a pair of symmetrical shell portions configured to be mateable to one another and collectively defining a pair of manifold outlet openings, a manifold inlet opening, and an air passageway extending therebetween.

13. The user interface of claim 12 further including:
a gas delivery member having a pair of inlet openings configured to be releasably coupleable to the pair of manifold outlet openings;
wherein:
the gas delivery member is configured as one of a nasal mask and a pair of nasal prongs, the nasal mask and nasal prongs being interchangeably connectable to the manifold member.

14. The user interface of claim 12 wherein:
the nasal mask and the pair of nasal prongs include a pair of flanges extending about a periphery of respective ones of the inlet openings;
the flanges being configured to be removably insertable into the manifold outlet openings.

15. The user interface of claim 12 wherein the manifold member includes at least one exhaust port.

16. A user interface adapted for delivering gas under pressure from a motor blower unit to a patient, the user interface comprising:
a manifold member having opposing end portions; and
at least one cheek pad extending laterally outwardly from the end portion and being configured to be freely orientatable relative thereto.

17. The user interface of claim 16 wherein the cheek pad is configured to be removably connectable to the manifold member.

18. The user interface of claim 16 further including:
at least one connector connectable to the manifold member and being configured to be freely orientatable relative thereto;
wherein:
the cheek pad is configured to be removably mountable to the connector.

19. The user interface of claim 18 wherein:
the connector includes a pair of apertures extending therethrough;
the cheek pad including a pair of pad mounting stems sized and configured to releasably engage the pair of apertures.

20. The user interface of claim 18 wherein:
the connector includes a connector ball formed on an end thereof;
the manifold member including a socket formed in an end portion thereof;
the socket being sized and configured to frictionally engage the connector ball.

21. Head gear for mounting a user interface to a patient's head, the head gear comprising:
a right-side assembly and a left-side assembly each including a side strap, a top head strap and a bottom head strap pivotally connectable to one another at a pivot joint; and
wherein:
each one of the head straps and side straps having a pivot end and an opposing free end;
the free ends of the side straps being adjustably connectable to opposing sides of the user interface;
the free ends of the top head straps being adjustably connectable to one another;
the free ends of the bottom head straps being adjustably connectable to one another.

22. The head gear of claim 21 wherein:
the free ends of the top head straps include complementary hook and loop fastener material such that the combined length of the top head straps is selectively adjustable;
the free ends of the bottom head straps include complementary hook and loop fastener material such that the combined length of the bottom head straps is selectively adjustable;
the free ends of each one of the side straps including hook and loop fastener material such that the length of each one of the side straps is selectively adjustable.

23. The head gear of claim 21 wherein:
each one of right-side and left-side assemblies includes a pair of the side straps pivotally connectable to the corresponding ones of the top and bottom head straps;
the free ends of each one of the side straps of each pair including complementary hook and loop fastener material such that the length of each one of the side straps is selectively adjustable.

24. The head gear of claim 21 wherein each one of the head and side straps is configured with straight side sections and being formed by die-cutting.

25. The head gear of claim 21 wherein the user interface is adapted for delivering gas under pressure from a motor blower unit to a patient, the user interface comprising:
a hollow manifold member having a pair of manifold outlet openings; and
a gas delivery member having a pair of inlet openings configured to be releasably coupleable to the pair of manifold outlet openings;
wherein:
the gas delivery member is configured as one of a nasal mask and a pair of nasal prongs, the nasal mask and nasal prongs be interchangeably connectable to the manifold member.
